(19) United States
(12) Patent Application Publication
(10) Pub. No.: US 2005/0014249 A1
(43) Pub. Date: Jan. 20, 2005

(54) Chromatographic analysis on optical bio-discs and methods relating thereto

(76) Inventors: Norbert Staimer, Lake Forest, CA (US); YihFar Chen, Kaohsiung City (TW)

Correspondence Address: KNOBBE MARTENS OLSON & BEAR LLP, 2040 MAIN STREET, FOURTEENTH FLOOR, IRVINE, CA, 92614, US

(21) Appl. No.: 10/785,376

(22) Filed: Sep. 20, 2004

Related U.S. Application Data
(60) Provisional application No. 60/449,192, filed on Feb. 21, 2003.

(51) Int. Cl. G11B 7/24; C12M 1/34; G01N 33/558

(52) U.S. Cl. 435/287.2; 436/514

(57) ABSTRACT

Preparation and analysis of biomedical samples is performed using an optical bio-disc system. More specifically, chromatography, including for example, affinity, size exclusion, reverse phase, and ion exchange may be performed using an optical bio-disc system. Ion exchange chromatography may include anion exchange, cation exchange, cation exchange linked immunoassays (CELIA), and anion exchange linked immunoassays, in conjunction with calorimetric and/or fluorescent detection and quantization using an optical analysis disc or optical bio-disc. Improved methods for preparing assays, methods for depositing the reagents required for the assays, discs for performing assays, and detection systems are described herein.

### REFERENCE TO RELATED APPLICATION

This application claims priority to U.S. Provisional Application No. 60/449,192, filed Feb. 21, 2003, which is hereby incorporated by reference in its entirety, including FIGS. 1-22C.

### BACKGROUND OF THE INVENTION

1. Field of the Invention

This invention relates in general to biochemical assays. More specifically, but without restriction to the particular embodiments hereinafter described in accordance with the best mode of practice, the present invention relates to methods and apparatus for chromatography based immunochemical assays performed on optical bio-discs and related detection systems.

2. Description of the Related Art

The detection and quantification of analytes in the blood or other body fluids are essential for diagnosis of diseases, elucidation of the pathogenesis, and for monitoring the response to drug treatment. Traditionally, diagnostic assays are performed in laboratories by trained technicians using complex apparatus. Performing these assays is usually time-consuming and costly. Thus, there is a significant need to make diagnostic assays and forensic assays of all types faster and more local to the end-user. Ideally, clinicians, patients, investigators, the military, other health care personnel, and consumers should be able to test themselves for the presence of certain risk factors or disease indicators in their systems, and to test for the presence of certain biological material at a crime scene or on a battlefield. At present, there are a number of medical diagnostic, silicon-based, devices with nucleic acids and/or proteins attached thereto that are commercially available or under development. These chips are not for use by the end-user, or for use by persons or entities lacking very specialized expertise and expensive equipment.

Commonly assigned U.S. Pat. No. 6,030,581 entitled “Laboratory in a Disk” issued Feb. 29, 2000 (the '581 patent) is hereby incorporated by reference in its entirety. The '581 patent discloses an apparatus that includes an optical disc, adapted to be read by an optical reader, which has a sector having a substantially self-contained assay system useful for localizing and detecting an analyte suspected of being in a sample. U.S. Pat. No. 5,993,665, issued Nov. 30, 1999 (the '665 patent) entitled “Quantitative Cell Analysis Methods Employing Magnetic Separation” discloses analysis of biological specimens in a fluid medium where the specimens are rendered magnetically responsive by immuno-specific binding with ferromagnetic colloid. The '665 patent is hereby incorporated by reference in its entirety.

### SUMMARY OF THE INVENTION

The present invention relates to performing chromatography, including for example, affinity, size exclusion, reverse phase, and ion exchange. Ion exchange chromatography may include anion exchange, cation exchange, cation exchange linked immunoassays (CELIA), and anion exchange linked immunoassays, in conjunction with colorimetric and/or fluorescent detection and quantitation using an optical analysis disc or optical bio-disc. The invention includes methods for preparing assays, methods for depositing the reagents required for the assays, discs for performing assays, and detection systems.

High pressure liquid chromatography (HPLC) and other types of chromatography is generally used to separate substances or analytes of interest having different physical properties and quantitate these analytes using UV/VIS, IR, luminescence, or fluorescence detection. Chromatographic instruments generally require costly equipment and maintenance and trained personnel to carry out complicated time-consuming tests. It is an object of the present invention to make possible a simple chromatography system for testing analytes, portable and for use by the end user.

The present invention includes methods for isolating and quantifying the concentration of an analyte of interest in a biological sample on optical bio-discs using calorimetric or fluorometric detection. Analytes may include, for example, Hemoglobin, glycated and non-glycated hemoglobin, and other isoforms of proteins. All reagents necessary for the assays may be immobilized on the optical disc prior to the assay. To perform the assay, the sample (preferably serum, but other types of body fluids could also be used) is loaded into the channel via the injection port. After injection, the port is sealed, the disc is spun, and the sample is moved through one or more micro-chromatographic matrices, by centrifugation, comprising different separation media including, for example, size exclusion and ion exchange matrices. The matrix may be formed from resins or beads, gels, or membranes. Once the analyte of interest is separated chromatographically, the analyte solution, containing the analyte of interest is then directed into an analysis chamber. The analysis chamber may contain detection reagents including, but not limited to, capture agents bound to the surface of a capture zone and signal antibodies conjugated with one or more reporters, both of which have affinity to different epitopes on the same analyte of interest. Reporters may include, but are not limited to, fluorophores, luminophores, microspheres, enzymes, and nanospheres. The analyte is incubated in the analysis chamber at a pre-determined temperature and time to allow sufficient binding of the analyte to the capture agent and binding of the signal antibodies to the analyte. After incubation the analysis chamber is washed to remove unbound signal antibodies and analytes. If the reporter used in the assay is a non-enzyme detectable reporter such as beads, then the analysis chamber may then be analysed for presence and amount of reporter beads using the disc reader. Otherwise, if an enzyme reporter is used, an enzyme substrate is added to the analysis chamber. The enzyme is allowed to catalize an enzyme-substrate reaction that produces a detectable signal such as color or fluorescence. The optical disc reader then quantifies the intensity of the color of fluorescence developed. After approximately 3 minutes of data collection and processing, the results of the assay are displayed on a computer monitor. Alternatively, an inherent enzymatic activity of the analyte itself may be advantageously used to produce a detectable signal. A non-limiting example of such an analyte is hemoglobin that has an inherent peroxidase activity. Thus capture and signal agents are not necessary with this method thereby allowing a one step assay method without the need for washing steps. In this method, the sample is loaded into the disc, ran through the matrix, and into the analysis chamber, as described above. The analysis chamber, in this method, would only contain the appropriate substrate, a peroxidase substrate like ABTS (2,2′-azino-di-[3-ethyl-benzthiazoline] sulfonic acid) may be used in conjunction with the hemoglobin analyte, for example. Once the signal is generated, the disc is then analyzed using the optical disc reader, as described above.

It should be noted that most diagnostic calorimetric assays in clinical laboratories are carried out at 37 degrees Celsius to facilitate and accelerate color development. For ease of operation, colorimetric assays performed on optical discs are optimized to run at ambient temperature. The optimization includes selection of enzyme sources, enzymes concentrations, and sample preparation.

Chromagen selection, in the calorimetric aspect of the present invention, is of critical importance in optimizing colorimetric assays for optical density measurements on bio-discs since chromagens need to be detected at specific wavelengths. CD-R type disc readers, for example, require chromagens that can be detected in the infrared region (750 nm to 800 nm). Other types of optical disc systems may be used in the present invention including DVD, DVD-R, fluorescent, phosphorescent, and any other similar optical disc reader. The amplitude of optical density measurements depends on the optical path length, the molar extinction coefficient of the chromagen and the concentration of the analyte of interest (Beer's law). To optimize the sensitivity of calorimetric assays on optical discs, several chromagens with high molar extinction coefficients at the wavelengths of interest have been identified and evaluated.

Chromagens suitable for colorimetric assays on CD-R type optical discs include, but are not limited to, N,N′-Bis(2-hydroxy-3-sulfopropyl) tolidine, disodium salt (SAT-3), N-(Carboxymethylaminocarbonyl)-4,4′-bis(dimethylamino)
-diphenylamine sodium salt (DA-64), 2,2′-azino-dimethylthiozoline-6-sulfonate (ABTS), Trinder's reagents N-Ethyl-N-(2-hydroxy-3-sulfopropyl)₃-methylaniline, sodium salt, dihydrate (TOOS) with the coupling reagent 3-(N-Methyl-N-phenylamino)-6-aminobenzenesulfonic acid, and sodium salt (NCP-11).

According to one aspect of the present invention, there are provided detection methods for quantifying the concentration of an analyte of interest in a biological sample on the bio-discs. The detection includes directing a beam of electromagnetic energy from a disc drive toward the capture field and analyzing electromagnetic energy returned from or transmitted through the capture field.

The optical density change, in the colorimetric assay aspect of the present invention, may be quantified by the optical disc reader by two related ways. These include measuring the change in light either reflected or transmitted. The disc may be referred to as reflective, transmissive, or some combination of reflective and transmissive. In a reflective disc, an incident light beam is focused onto the disc (typically at a reflective surface where information is encoded), reflected, and returned through optical elements to a detector on the same side of the disc as the light source. In a transmissive disc, light passes through the disc (or portions thereof) to a detector on the other side of the disc from the light source. In a transmissive portion of a disc, some light may also be reflected and detected as reflected light. Different detection systems are used for different types of bio-discs (top versus bottom detector).

The conversion of data captured by the CD reader into meaningful concentration units is mediated via data processing software specific for the assay of interest.

The apparatus and methods in embodiments of the present invention can be designed for use by an end-user, inexpensively, without specialized expertise and expensive equipment. The system can be made portable, and thus usable in remote locations where traditional diagnostic equipment may not generally be available.

Alternatively, fluorescent assays can be carried out to quantify the concentration of an analyte of interest in a biological sample on the optical discs. In this case, the energy source in the disc drive preferably has a wavelength controllable light source and a detector that is or can be made specific to a particular wavelength. In yet another alternative, a disc drive can be made with a specific light source and detector to produce a dedicated device, in which case the source may only need fine-tuning.

Analysis of biological fluids aimed at the quantitative and qualitative determination of substances associated with a wide variety of physiological disorders, bioresearch, proteomics, environmental studies, agriculture, and food industry, relies on specific binding assays from which the immunoassay plays a dominant role. The outstanding specificity and sensitivity for quantitative determination of an almost limitless number of analytes in practically any milieu, and the ability to miniaturize and adapt to automation makes them ideal tools for routine assays.

Antibody binding techniques are based on the interaction of a binding antibody, receptor, or other binding proteins with an antigen or a specific ligand molecule and the formation of an antibody-antigen or receptor-ligand complex. By changing certain conditions a binding assay can be designed to determine either an analyte, ligand, or target binding reagent or an antibody of interest. The steps are similar but the assay configuration provides results pertinent to the antigen or antibody of interest.

1. Capture Probe Binding and Sample Application

When a sample is injected into a micro-channel, fluidic circuit, or flow channel on an optical bio-disc, the target agent including, for example, target antigen or antibody, binds to a capture probe bound in a capture or target zone on a solid support such as a disc substrate. The capture probe may be an antigen recognized by the target antibody or an antibody or receptor with specific affinity to the target antigen or ligand. Following the binding step, unbound target agent is removed through a wash step. It should be understood that various techniques, procedures and chemistries, know in the art, may be used to bind the capture probe onto a solid support including, but not limited to, direct covalent binding of probes onto a metallic or activated surface, passive adsorption, and through cross-linking reagents.

Further details relating to surface chemistries used to bind probes onto solid support are disclosed in, for example, the above incorporated commonly assigned co-pending U.S. Provisional Application Ser. No. 60/353,770 entitled “Capture Layer Assemblies Including Metal Layer for Immobilization of Receptor Molecules and Related Optical Assay Discs” filed Jan. 30, 2002; and U.S. Provisional Application Ser. No. 60/353,745 entitled “Capture Layer Assemblies Including Polymer Substrates for Immobilization of Receptor Molecules and Related Optical Assay Discs” filed Jan. 30, 2002.

In addition to surface chemistries for attaching capture probes, blocking agents may be used to block areas within the capture or target zone and the flow channel where capture probes are not bound (non-capture areas) to prevent non-specific binding of the target or analyte, signal probes, and reporters onto these areas. Blocking agents include, but are not limited to proteins such as BSA, gelatin, sugars such as sucrose, detergents such as tween-20, genetic material such as sheared salmon sperm DNA, and polyvinyl alcohol.

2. Signal Generation

Signal is generated from tags or labels attached to signal or reporter agents or probes that have specific affinity to a target agent. Signal agents or probes may include, for example, signal antibodies or signal ligands, tagged with microspheres, sub-micron nanospheres, or enzymes. The microspheres or nanospheres may be fluorescent labeled (fluospheres), phosphorescent, luminecent, or chemiluminescent. The microspheres or nanospheres may also carry different chemical functionalities including, for example, carboxyl, amino, aldehyde, and hydrazine functional groups. These functional groups may facilitate binding of the signal agent. The enzyme may facilitate a chemical reaction that produces fluorescence, color, or a detectable signal in the presence of a suitable substrate. For example, conjugated horseradish peroxidase (HRP; Pierce, Rockford, Ill.) may be used with the substrate 3,3,5,5-tetramethylbenzidine (TMB; Calbiochem cat. no. 613548, CAS-54827-17-7) in the presence of hydrogen peroxide to produce an insoluble precipitate. Horseradish peroxidase can also be used in conjunction with CN/DAB (4-chloronaphthol/3,3′-diaminobenzidine, tetrahydrochloride), 4-CN (4-chloro-1-napthol), AEC (3-amino-9-ethyl carbazol) and DAB (3,3-diaminobenzidine tetrahydrochloride) to form insoluble precipitates. Similarly, the enzyme alkaline phosphatase (AP) can be used with the substrate bromochloroindolylphosphate in the practice of the present invention. Other suitable enzyme/substrate combinations will be apparent to those of skill in the art.

3. Detection

The signal from the microspheres or the enzyme reaction can be read with the optical bio-disc readers developed to be utilized in conjunction herewith. Either a bottom detector on a disc with a reflective cover, or a top detector with a transmissive disc may be employed as the optical bio-disc reader for the assay and disc inventions disclosed herein.

(a) Disc Implementation

The assays and methods of the present invention may be advantageously implemented on an analysis disc, modified optical disc, or bio-disc. The bio-disc may include a flow channel having target or capture zone, a return channel in fluid communication therewith, a mixing chamber in fluid communication with the flow channel, and in some embodiments a waste reservoir in fluid communication with the flow channel.

The bio-disc may be implemented on an optical disc including an information encoding format such as CD, CD-R, or DVD or a modified version thereof. The bio-disc may include encoded information for performing, controlling, and post-processing the test or assay. For example, such encoded information may be directed to controlling the rotation rate of the disc, incubation time, incubation temperature, and/or specific steps of the assay. Depending on the test, assay, or investigational protocol, the rotation rate may be variable with intervening or consecutive sessions of acceleration, constant speed, and deceleration. These sessions may be closely controlled both as to speed and time of rotation to provide, for example, mixing, agitation, or separation of fluids and suspensions with agents, reagents, DNA, RNA, antigen, antibodies, ligands, and receptors.

(b) Drive Implementation

A bio-disc drive assembly or reader may be employed to rotate the disc, read and process any encoded information stored on the disc, and analyze the samples in the flow channel of the bio-disc. The bio-disc drive is thus provided with a motor for rotating the bio-disc, a controller for controlling the rate of rotation of the disc, a processor for processing return signals from the disc, and an analyzer for analyzing the processed signals. The drive may include software specifically developed for performing the assays disclosed herein.

The rotation rate of the motor is controlled to achieve the desired rotation of the disc. The bio-disc drive assembly may also be utilized to write information to the bio-disc either before or after the test material in the flow channel and target or capture zone is interrogated by the read beam of the drive and analyzed by the analyzer. The bio-disc may include encoded information for controlling the rotation rate of the disc, providing processing information specific to the type of test to be conducted, and for displaying the results on a display monitor associated with the bio-drive in accordance with the assay methods relating hereto.

### BRIEF DESCRIPTION OF THE DRAWINGS

Further objects of the present invention together with additional features contributing thereto and advantages accruing therefrom will be apparent from the following description of the preferred embodiments of the invention which are shown in the accompanying drawing figures with like reference numerals indicating like components throughout, wherein:

FIG. 1 is a pictorial representation of a bio-disc system;

FIG. 2 is an exploded perspective view of a reflective bio-disc;

FIG. 3 is a top plan view of the disc shown in FIG. 2;

FIG. 4 is a perspective view of the disc illustrated in FIG. 2 with cut-away sections showing the different layers of the disc;

FIG. 5 is an exploded perspective view of a transmissive bio-disc;

FIG. 6 is a perspective view representing the disc shown in FIG. 5 with a cut-away section illustrating the functional aspects of a semi-reflective layer of the disc;

FIG. 7 is a graphical representation showing the relationship between thickness and transmission of a thin gold film;

FIG. 8 is a top plan view of the disc shown in FIG. 5;

FIG. 9 is a perspective view of the disc illustrated in FIG. 5 with cut-away sections showing the different layers of the disc including the type of semi-reflective layer shown in FIG. 6;

FIG. 10 is a perspective and block diagram representation illustrating the system of FIG. 1 in more detail;

FIG. 11 is a partial cross sectional view taken perpendicular to a radius of the reflective optical bio-disc illustrated in FIGS. 2, 3, and 4 showing a flow channel formed therein;

FIG. 12 is a partial cross sectional view taken perpendicular to a radius of the transmissive optical bio-disc illustrated in FIGS. 5, 8, and 9 showing a flow channel formed therein and a top detector;

FIG. 13 is a partial longitudinal cross sectional view of the reflective optical bio-disc shown in FIGS. 2, 3, and 4 illustrating a wobble groove formed therein;

FIG. 14 is a partial longitudinal cross sectional view of the transmissive optical bio-disc illustrated in FIGS. 5, 8, and 9 showing a wobble groove formed therein and a top detector;

FIG. 15 is a view similar to FIG. 11 showing the entire thickness of the reflective disc and the initial refractive property thereof;

FIG. 16 is a view similar to FIG. 12 showing the entire thickness of the transmissive disc and the initial refractive property thereof;

FIG. 17A is an exploded perspective view of a reflective bio-disc incorporating equi-radial channels of the present invention;

FIG. 17B is a top plan view of the disc shown in FIG. 17A;

FIG. 17C is a perspective view of the disc illustrated in FIG. 17A with cut-away sections showing the different layers of the equi-radial reflective disc;

FIG. 18A is an exploded perspective view of a transmissive bio-disc utilizing the e-radial channels of the present invention;

FIG. 18B is a top plan view of the disc shown in FIG. 18A;

FIG. 18C is a perspective view of the disc illustrated in FIG. 18A with cut-away sections showing the different layers of this embodiment of the equi-radial transmissive bio-disc;

FIG. 19 is a pictorial representation of images derived from a transmissive optical bio-disc showing differences in signal derived from various concentrations of the hemoglobin;

FIG. 20 is a graphical representation of a dose response curve generated using the optical bio-disc system of the present invention;

FIG. 21 is a top plan view of another embodiment of the optical bio-disc having a micro-chromatographic matrix in a fluidic circuit;

FIG. 22A are top plan views of various layers of a chromatographic optical bio-disc of the present invention;

FIG. 22B is an exploded perspective view of the chromatographic optical bio-disc of FIG. 22A; and

FIG. 22C is a partial cross sectional view taken perpendicular to a radius of the optical bio-disc illustrated in FIG. 22B showing the direction sample flow within the fluidic circuit.

### DETAILED DESCRIPTION OF THE PREFERRED EMBODIMENT

The present invention relates in general to preparation of biomedical samples and analysis of same using an optical bio-disc system. More specifically, this invention is directed to performing chromatography, including for example, affinity, size exclusion, reverse phase, and ion exchange. Ion exchange chromatography may include anion exchange, cation exchange, cation exchange linked immunoassays (CELIA), and anion exchange linked immunoassays, in conjunction with calorimetric and/or fluorescent detection and quantitation using an optical analysis disc or optical bio-disc. The invention includes methods for preparing assays, methods for depositing the reagents required for the assays, discs for performing assays, and detection systems. Each of the aspects of the present invention is discussed below in further detail.

Drive System and Related Discs

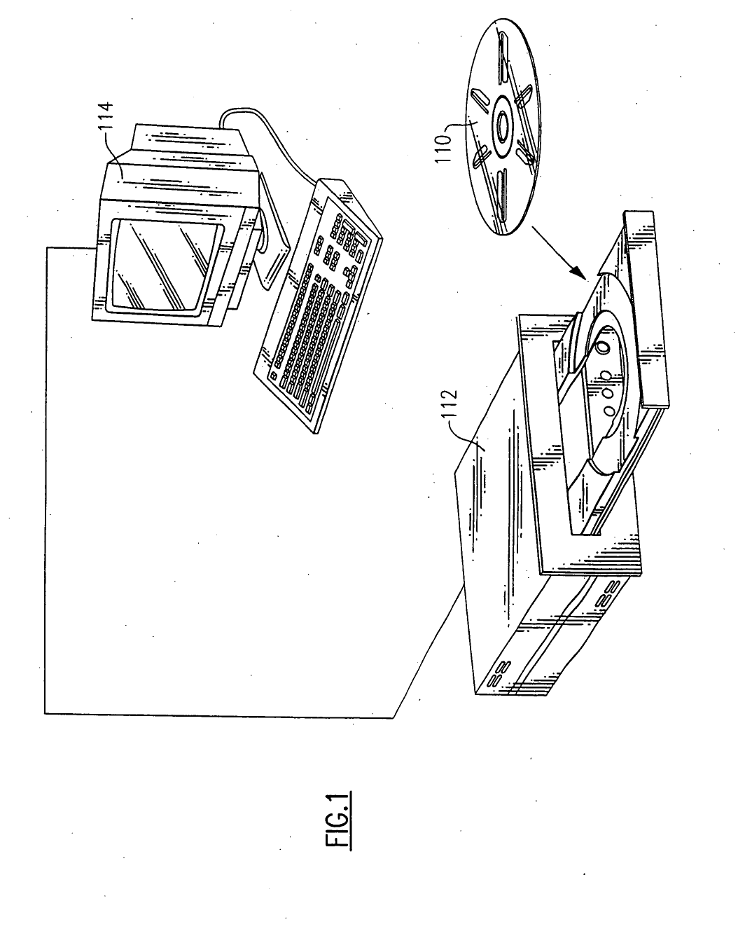

FIG. 1 is a perspective view of an optical bio-disc 110 according to the present invention as implemented to conduct the cell counts and differential cell counts disclosed herein. The present optical bio-disc 110 is shown in conjunction with an optical disc drive 112 and a display monitor 114. Further details relating to this type of disc drive and disc analysis system are disclosed in commonly assigned and co-pending U.S. patent application Ser. No. 10/008,156 entitled “Disc Drive System and Methods for Use with Bio-discs” filed Nov. 9, 2001 and U.S. patent application Ser. No. 10/043,688 entitled “Optical Disc Analysis System Including Related Methods For Biological and Medical Imaging” filed Jan. 10, 2002, both of which are herein incorporated by reference.

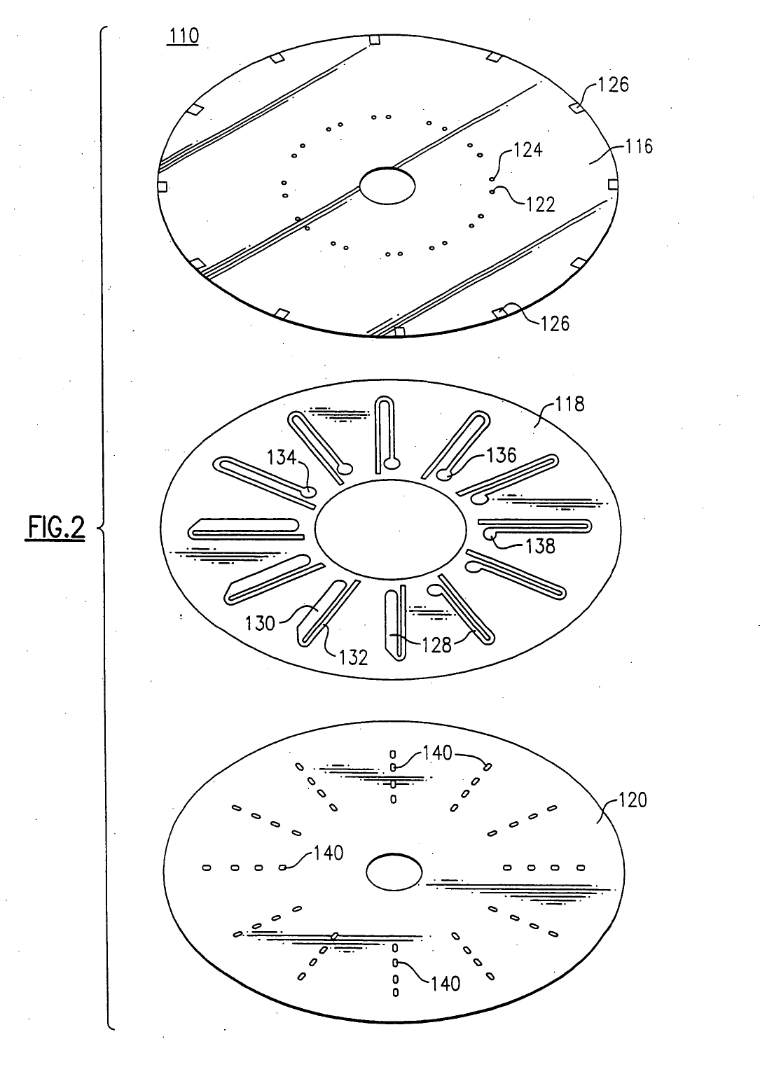

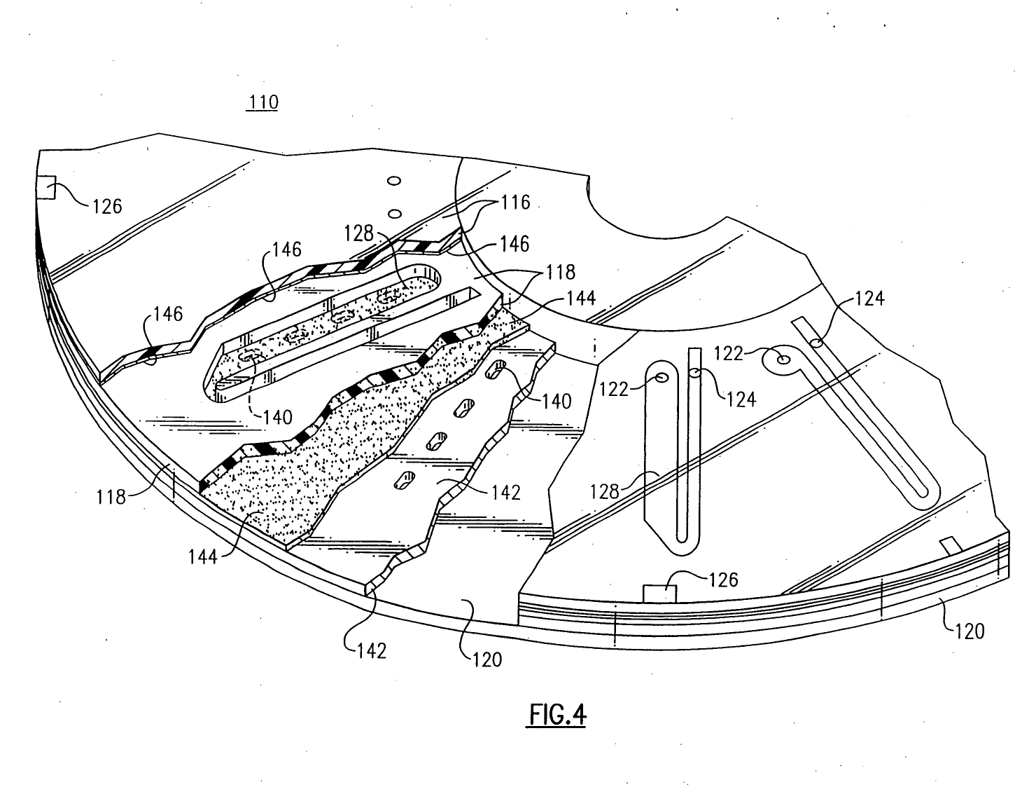

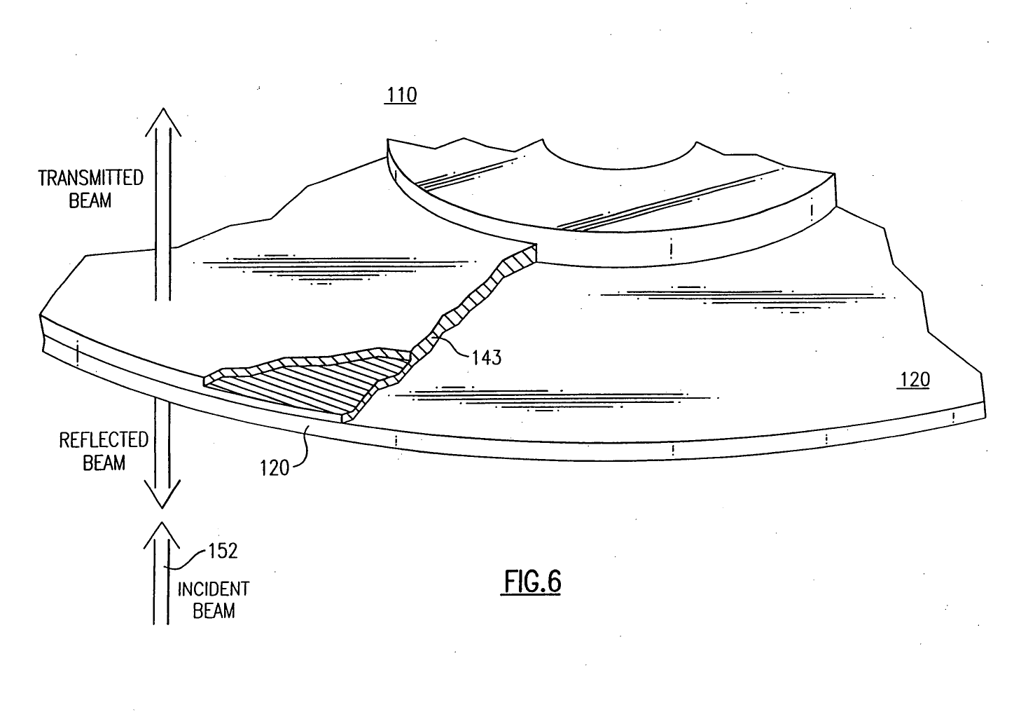

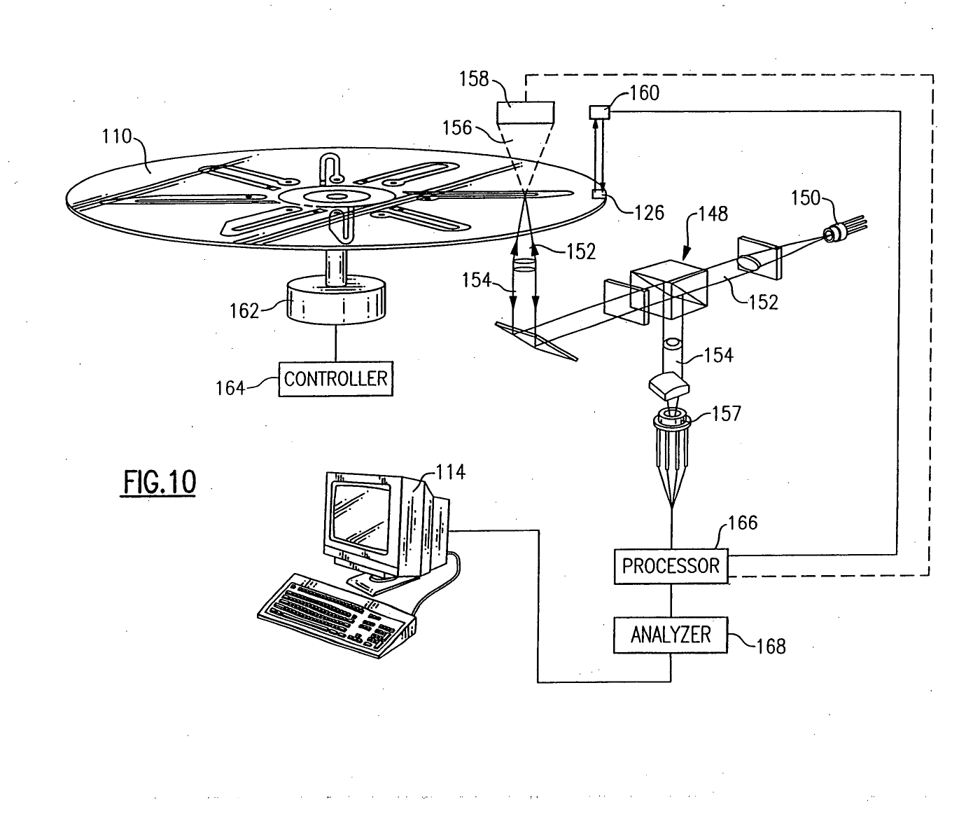

FIG. 2 is an exploded perspective view of the principal structural elements of one embodiment of the optical bio-disc 110. FIG. 2 is an example of a reflective zone optical bio-disc 110 (hereinafter “reflective disc”) that may be used in the present invention. The principal structural elements include a cap portion 116, an adhesive member or channel layer 118, and a substrate 120. The cap portion 116 includes one or more inlet ports 122 and one or more vent ports 124. The cap portion 116 may be formed from polycarbonate and is preferably coated with a reflective surface 146 (FIG. 4) on the bottom thereof as viewed from the perspective of FIG. 2. In the preferred embodiment, trigger marks or markings 126 are included on the surface of the reflective layer 142 (FIG. 4). Trigger markings 126 may include a clear window in all three layers of the bio-disc, an opaque area, or a reflective or semi-reflective area encoded with information that sends data to a processor 166, as shown FIG. 10, that in turn interacts with the operative functions of the interrogation or incident beam 152, FIGS. 6 and 10.

The second element shown in FIG. 2 is an adhesive member or channel layer 118 having fluidic circuits 128 or U-channels formed therein. The fluidic circuits 128 are formed by stamping or cutting the membrane to remove plastic film and form the shapes as indicated. Each of the fluidic circuits 128 includes a flow channel 130 and a return channel 132. Some of the fluidic circuits 128 illustrated in FIG. 2 include a mixing chamber 134. Two different types of mixing chambers 134 are illustrated. The first is a symmetric mixing chamber 136 that is symmetrically formed relative to the flow channel 130. The second is an off-set mixing chamber 138. The off-set mixing chamber 138 is formed to one side of the flow channel 130 as indicated.

The third element illustrated in FIG. 2 is a substrate 120 including target or capture zones 140. The substrate 120 is preferably made of polycarbonate and has a reflective layer 142 deposited on the top thereof, FIG. 4. The target zones 140 are formed by removing the reflective layer 142 in the indicated shape or alternatively in any desired shape. Alternatively, the target zone 140 may be formed by a masking technique that includes masking the target zone 140 area before applying the reflective layer 142. The reflective layer 142 may be formed from a metal such as aluminum or gold.

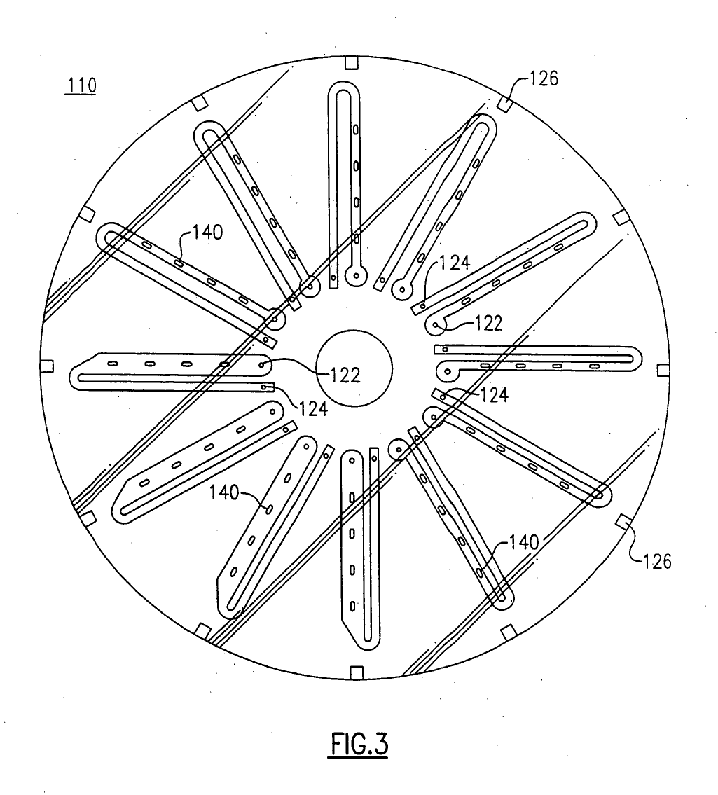

FIG. 3 is a top plan view of the optical bio-disc 110 illustrated in FIG. 2 with the reflective layer 142 on the cap portion 116 shown as transparent to reveal the fluidic circuits 128, the target zones 140, and trigger markings 126 situated within the disc.

FIG. 4 is an enlarged perspective view of the reflective zone type optical bio-disc 110 according to one embodiment of the present invention. This view includes a portion of the various layers thereof, cut away to illustrate a partial sectional view of each principal layer, substrate, coating, or membrane. FIG. 4 shows the substrate 120 that is coated with the reflective layer 142. An active layer 144 is applied over the reflective layer 142. In the preferred embodiment, the active layer 144 may be formed from polystyrene. Alternatively, polycarbonate, gold, activated glass, modified glass, or modified polystyrene, for example, polystyrene-co-maleic anhydride, may be used. In addition, hydrogels can be used. Alternatively as illustrated in this embodiment, the plastic adhesive member 118 is applied over the active layer 144. The exposed section of the plastic adhesive member 118 illustrates the cut out or stamped U-shaped form that creates the fluidic circuits 128. The final principal structural layer in this reflective zone embodiment of the present bio-disc is the cap portion 116. The cap portion 116 includes the reflective surface 146 on the bottom thereof. The reflective surface 146 may be made from a metal such as aluminum or gold.

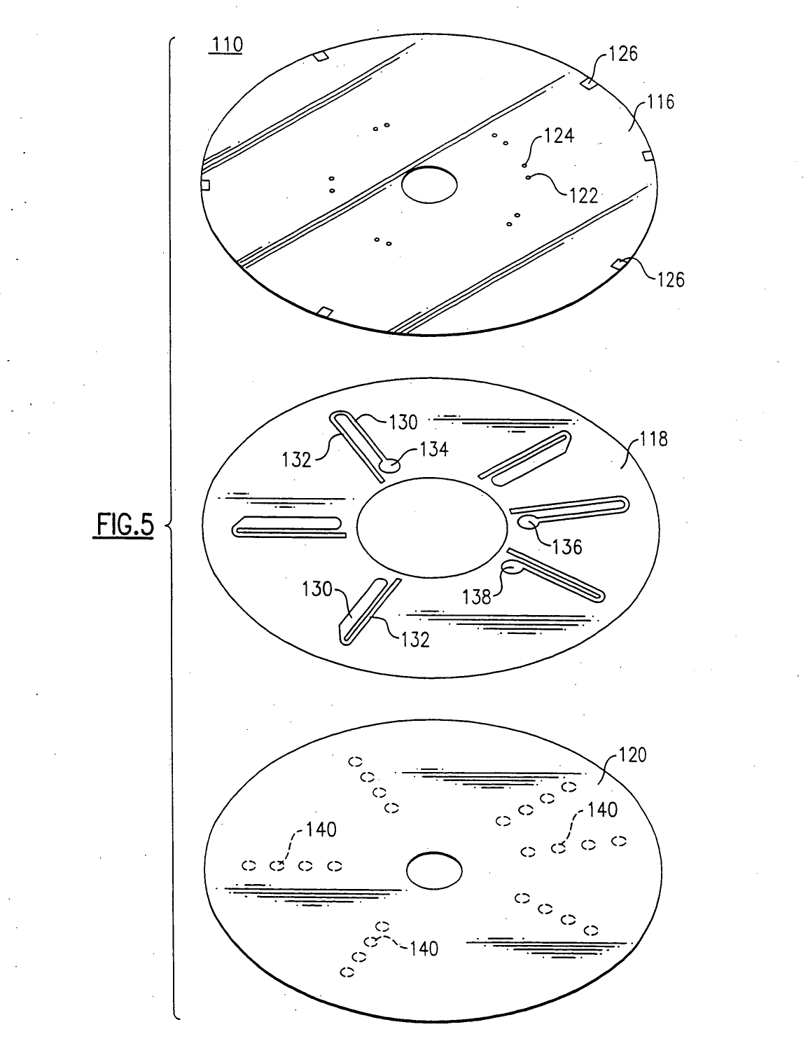

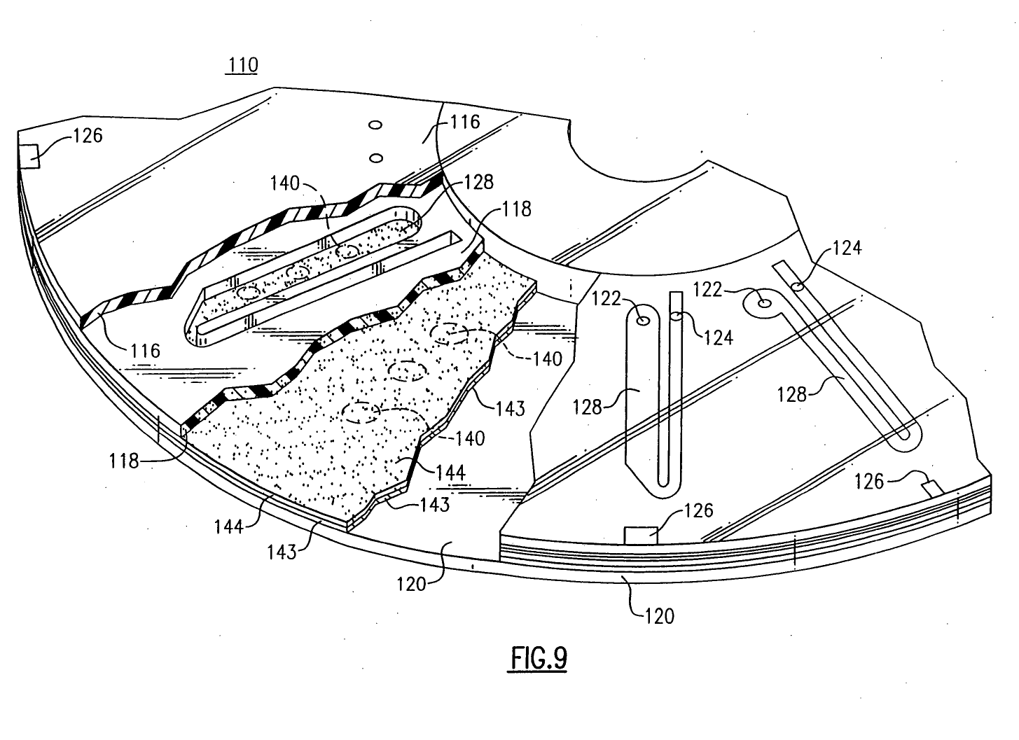

Referring now to FIG. 5, there is shown an exploded perspective view of the principal structural elements of a transmissive type of optical bio-disc 110 according to the present invention. The principal structural elements of the transmissive type of optical bio-disc 110 similarly include the cap portion 116, the adhesive or channel member 118, and the substrate 120 layer. The cap portion 116 includes one or more inlet ports 122 and one or more vent ports 124. The cap portion 116 may be formed from a polycarbonate layer. Optional trigger markings 126 may be included on the surface of a thin semi-reflective layer 143, as best illustrated in FIGS. 6 and 9. Trigger markings 126 may include a clear window in all three layers of the bio-disc, an opaque area, or a reflective or semi-reflective area encoded with information that sends data to the processor 166, FIG. 10, which in turn interacts with the operative functions of the interrogation beam 152, FIGS. 6 and 10.

The second element shown in FIG. 5 is the adhesive member or channel layer 118 having fluidic circuits 128 or U-channels formed therein. The fluidic circuits 128 are formed by stamping or cutting the membrane to remove plastic film and form the shapes as indicated. Each of the fluidic circuits 128 includes the flow channel 130 and the return channel 132. Some of the fluidic circuits 128 illustrated in FIG. 5 include the mixing chamber 134. Two different types of mixing chambers 134 are illustrated. The first is the symmetric mixing chamber 136 that is symmetrically formed relative to the flow channel 130. The second is the off-set mixing chamber 138. The off-set mixing chamber 138 is formed to one side of the flow channel 130 as indicated.

The third element illustrated in FIG. 5 is the substrate 120, which may include the target or capture zones 140. The substrate 120 is preferably made of polycarbonate and has the thin semi-reflective layer 143 deposited on the top thereof, FIG. 6. The semi-reflective layer 143 associated with the substrate 120 of the disc 110 illustrated in FIGS. 5 and 6 is significantly thinner than the reflective layer 142 on the substrate 120 of the reflective disc 110 illustrated in FIGS. 2, 3 and 4. The thinner semi-reflective layer 143 allows for some transmission of the interrogation beam 152 through the structural layers of the transmissive disc as shown in FIGS. 6 and 12. The thin semi-reflective layer 143 may be formed from a metal such as aluminum or gold.

FIG. 6 is an enlarged perspective view of the substrate 120 and semi-reflective layer 143 of the transmissive embodiment of the optical bio-disc 110 illustrated in FIG. 5. The thin semi-reflective layer 143 may be made from a metal such as aluminum or gold. In the preferred embodiment, the thin semi-reflective layer 143 of the transmissive disc illustrated in FIGS. 5 and 6 is approximately 100-300 Å thick and does not exceed 400 Å. This thinner semi-reflective layer 143 allows a portion of the incident or interrogation beam 152 to penetrate and pass through the semi-reflective layer 143 to be detected by a top detector 158, FIGS. 10 and 12, while some of the light is reflected or returned back along the incident path. As indicated below, Table 1 presents the reflective and transmissive characteristics of a gold film relative to the thickness of the film. The gold film layer is fully reflective at a thickness greater than 800 Å. While the threshold density for transmission of light through the gold film is approximately 400 Å.

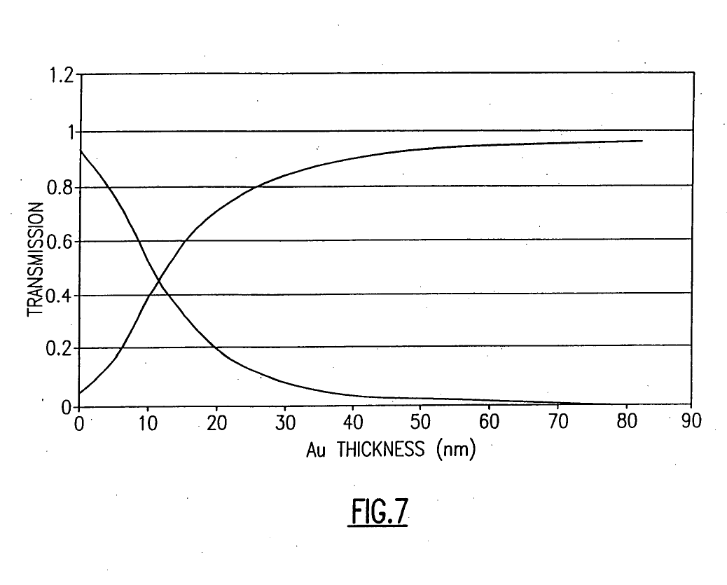

In addition to Table 1, FIG. 7 provides a graphical representation of the inverse relationship of the reflective and transmissive nature of the thin semi-reflective layer 143 based upon the thickness of the gold. Reflective and transmissive values used in the graph illustrated in FIG. 7 are absolute values.

TABLE 1
Au Film Reflection and Transmission (Absolute Values)

| Thickness | | | |
|---|---|---|---|
| (Angstroms) | Thickness (nm) | Reflectance | Transmittance |
| 0 | 0 | 0.0505 | 0.9495 |
| 50 | 5 | 0.1683 | 0.7709 |
| 100 | 10 | 0.3981 | 0.5169 |
| 150 | 15 | 0.5873 | 0.3264 |
| 200 | 20 | 0.7142 | 0.2057 |
| 250 | 25 | 0.7959 | 0.1314 |
| 300 | 30 | 0.8488 | 0.0851 |
| 350 | 35 | 0.8836 | 0.0557 |
| 400 | 40 | 0.9067 | 0.0368 |
| 450 | 45 | 0.9222 | 0.0244 |
| 500 | 50 | 0.9328 | 0.0163 |
| 550 | 55 | 0.9399 | 0.0109 |
| 600 | 60 | 0.9448 | 0.0073 |
| 650 | 65 | 0.9482 | 0.0049 |
| 700 | 70 | 0.9505 | 0.0033 |
| 750 | 75 | 0.9520 | 0.0022 |
| 800 | 80 | 0.9531 | 0.0015 |

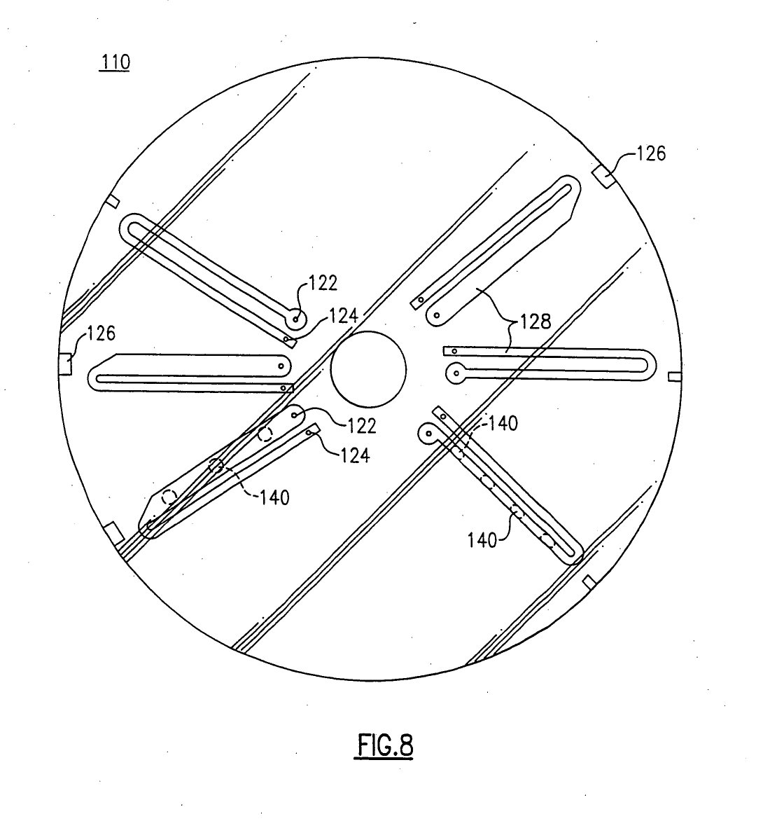

With reference next to FIG. 8, there is shown a top plan view of the transmissive type optical bio-disc 110 illustrated in FIGS. 5 and 6 with the transparent cap portion 116 revealing the fluidic channels, the trigger markings 126, and the target zones 140 as situated within the disc.

FIG. 9 is an enlarged perspective view of the optical bio-disc 110 according to the transmissive disc embodiment of the present invention. The disc 110 is illustrated with a portion of the various layers thereof cut away to show a partial sectional view of each principal layer, substrate, coating, or membrane. FIG. 9 illustrates a transmissive disc format with the clear cap portion 116, the thin semi-reflective layer 143 on the substrate 120, and trigger markings 126. In this embodiment, trigger markings 126 include opaque material placed on the top portion of the cap. Alternatively the trigger marking 126 may be formed by clear, non-reflective windows etched on the thin reflective layer 143 of the disc, or any mark that absorbs or does not reflect the signal coming from the trigger detector 160, FIG. 10. FIG. 9 also shows the target zones 140 formed by marking the designated area in the indicated shape or alternatively in any desired shape. Markings to indicate target zone 140 may be made on the thin semi-reflective layer 143 on the substrate 120 or on the bottom portion of the substrate 120 (under the disc). Alternatively, the target zones 140 may be formed by a masking technique that includes masking the entire thin semi-reflective layer 143 except the target zones 140. In this embodiment, target zones 140 may be created by silk screening ink onto the thin semi-reflective layer 143. In the transmissive disc format illustrated in FIGS. 5, 8, and 9, the target zones 140 may alternatively be defined by address information encoded on the disc. In this embodiment, target zones 140 do not include a physically discernable edge boundary.

With continuing reference to FIG. 9, an active layer 144 is illustrated as applied over the thin semi-reflective layer 143. In the preferred embodiment, the active layer 144 is a 10 to 200 μm thick layer of 2% polystyrene. Alternatively, polycarbonate, gold, activated glass, modified glass, or modified polystyrene, for example, polystyrene-co-maleic anhydride, may be used. In addition, hydrogels can be used. As illustrated in this embodiment, the plastic adhesive member 118 is applied over the active layer 144. The exposed section of the plastic adhesive member 118 illustrates the cut out or stamped U-shaped form that creates the fluidic circuits 128.

The final principal structural layer in this transmissive embodiment of the present bio-disc 110 is the clear, non-reflective cap portion 116 that includes inlet ports 122 and vent ports 124.

Referring now to FIG. 10, there is a representation in perspective and block diagram illustrating optical components 148, a light source 150 that produces the incident or interrogation beam 152, a return beam 154, and a transmitted beam 156. In the case of the reflective bio-disc illustrated in FIG. 4, the return beam 154 is reflected from the reflective surface 146 of the cap portion 116 of the optical bio-disc 110. In this reflective embodiment of the present optical bio-disc 110, the return beam 154 is detected and analyzed for the presence of signal elements by a bottom detector 157. In the transmissive bio-disc format, on the other hand, the transmitted beam 156 is detected, by a top detector 158, and is also analyzed for the presence of signal elements. In the transmissive embodiment, a photo detector may be used as a top detector 158.

FIG. 10 also shows a hardware trigger mechanism that includes the trigger markings 126 on the disc and a trigger detector 160. The hardware triggering mechanism is used in both reflective bio-discs (FIG. 4) and transmissive bio-discs (FIG. 9). The triggering mechanism allows the processor 166 to collect data only when the interrogation beam 152 is on a respective target zone 140. Furthermore, in the transmissive bio-disc system, a software trigger may also be used. The software trigger uses the bottom detector to signal the processor 166 to collect data as soon as the interrogation beam 152 hits the edge of a respective target zone 140. FIG. 10 further illustrates a drive motor 162 and a controller 164 for controlling the rotation of the optical bio-disc 110. FIG. 10 also shows the processor 166 and analyzer 168 implemented in the alternative for processing the return beam 154 and transmitted beam 156 associated the transmissive optical bio-disc.

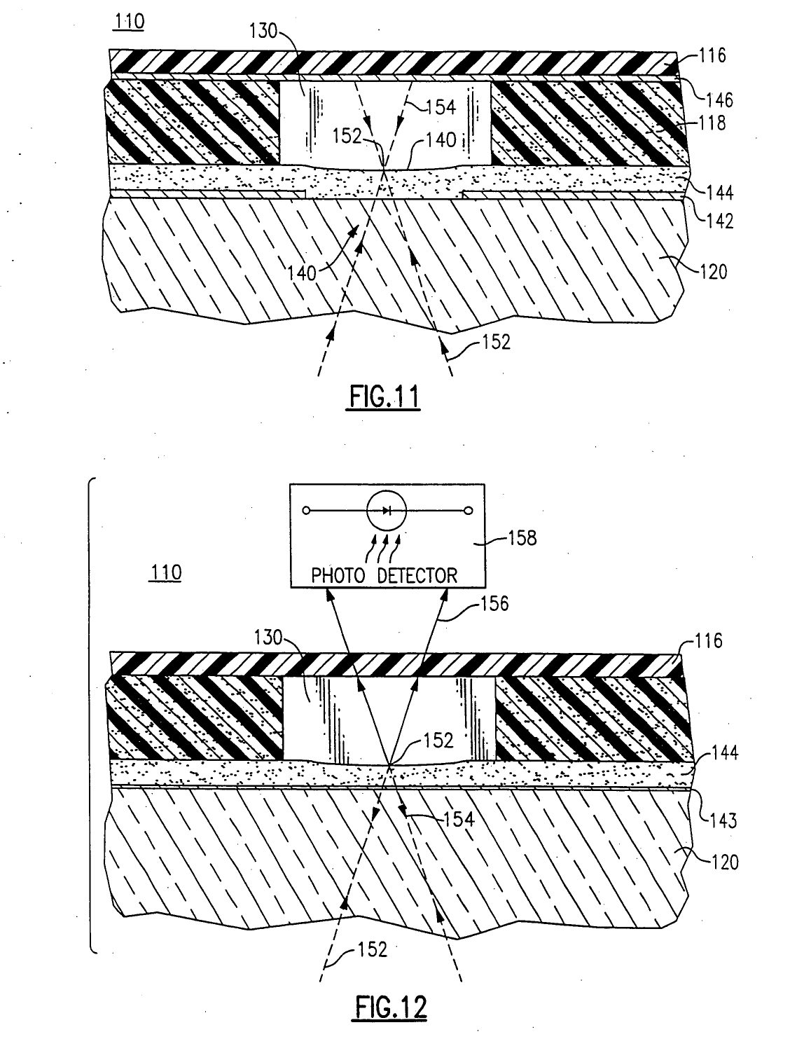

As shown in FIG. 11, there is presented a partial cross sectional view of the reflective disc embodiment of the optical bio-disc 110 according to the present invention. FIG. 11 illustrates the substrate 120 and the reflective layer 142. As indicated above, the reflective layer 142 may be made from a material such as aluminum, gold or other suitable reflective material. In this embodiment, the top surface of the substrate 120 is smooth. FIG. 11 also shows the active layer 144 applied over the reflective layer 142. As also shown in FIG. 11, the target zone 140 is formed by removing an area or portion of the reflective layer 142 at a desired location or, alternatively, by masking the desired area prior to applying the reflective layer 142. As further illustrated in FIG. 11, the plastic adhesive member 118 is applied over the active layer 144. FIG. 11 also shows the cap portion 116 and the reflective surface 146 associated therewith. Thus when the cap portion 116 is applied to the plastic adhesive member 118 including the desired cutout shapes, flow channel 130 is thereby formed. As indicated by the arrowheads shown in FIG. 11, the path of the incident beam 152 is initially directed toward the substrate 120 from below the disc 110. The incident beam then focuses at a point proximate the reflective layer 142. Since this focusing takes place in the target zone 140 where a portion of the reflective layer 142 is absent, the incident continues along a path through the active layer 144 and into the flow channel 130. The incident beam 152 then continues upwardly traversing through the flow channel to eventually fall incident onto the reflective surface 146. At this point, the incident beam 152 is returned or reflected back along the incident path and thereby forms the return beam 154.

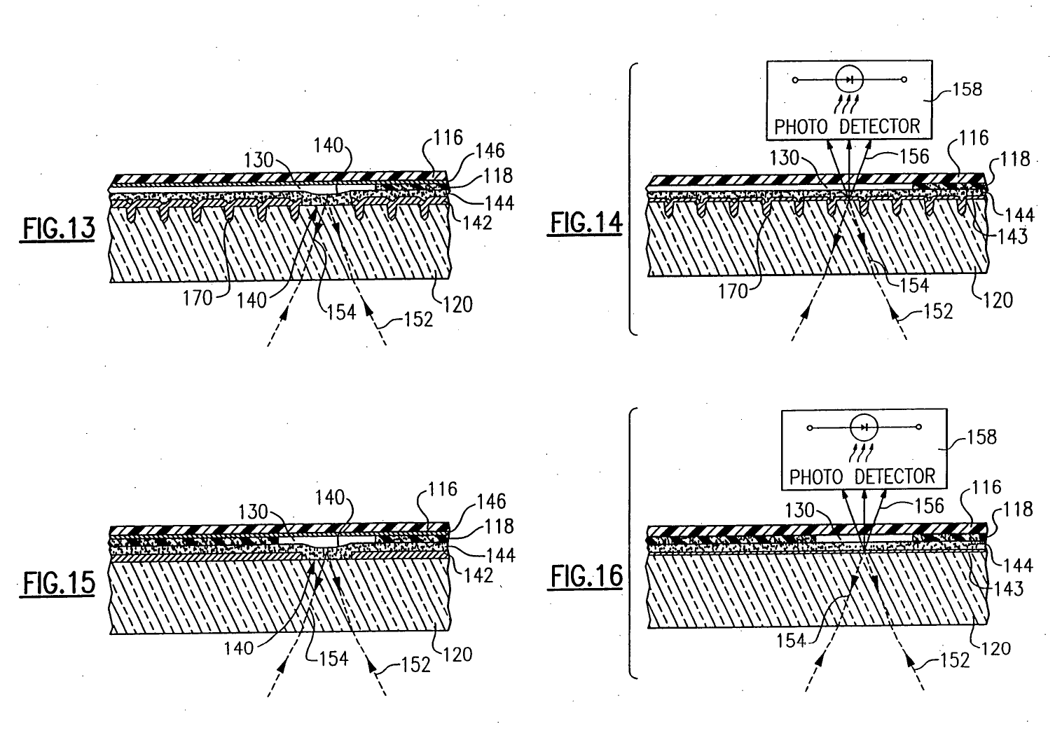

FIG. 12 is a partial cross sectional view of the transmissive embodiment of the bio-disc 110 according to the present invention. FIG. 12 illustrates a transmissive disc format with the clear cap portion 116 and the thin semi-reflective layer 143 on the substrate 120. FIG. 12 also shows the active layer 144 applied over the thin semi-reflective layer 143. In the preferred embodiment, the transmissive disc has the thin semi-reflective layer 143 made from a metal such as aluminum or gold approximately 100-300 Angstroms thick and does not exceed 400 Angstroms. This thin semi-reflective layer 143 allows a portion of the incident or interrogation beam 152, from the light source 150, FIG. 10, to penetrate and pass upwardly through the disc to be detected by a top detector 158, while some of the light is reflected back along the same path as the incident beam but in the opposite direction. In this arrangement, the return or reflected beam 154 is reflected from the semi-reflective layer 143. Thus in this manner, the return beam 154 does not enter into the flow channel 130. The reflected light or return beam 154 may be used for tracking the incident beam 152 on pre-recorded information tracks formed in or on the semi-reflective layer 143 as described in more detail in conjunction with FIGS. 13 and 14. In the disc embodiment illustrated in FIG. 12, a physically defined target zone 140 may or may not be present. Target zone 140 may be created by direct markings made on the thin semi-reflective layer 143 on the substrate 120. These marking may be formed using silk screening or any equivalent method. In the alternative embodiment where no physical indicia are employed to define a target zone (such as, for example, when encoded software addressing is utilized) the flow channel 130 in effect may be employed as a confined target area in which inspection of an investigational feature is conducted.

FIG. 13 is a cross sectional view taken across the tracks of the reflective disc embodiment of the bio-disc 110 according to the present invention. This view is taken longitudinally along a radius and flow channel of the disc. FIG. 13 includes the substrate 120 and the reflective layer 142. In this embodiment, the substrate 120 includes a series of grooves 170. The grooves 170 are in the form of a spiral extending from near the center of the disc toward the outer edge. The grooves 170 are implemented so that the interrogation beam 152 may track along the spiral grooves 170 on the disc. This type of groove 170 is known as a “wobble groove”. A bottom portion having undulating or wavy sidewalls forms the groove 170, while a raised or elevated portion separates adjacent grooves 170 in the spiral. The reflective layer 142 applied over the grooves 170 in this embodiment is, as illustrated, conformal in nature. FIG. 13 also shows the active layer 144 applied over the reflective layer 142. As shown in FIG. 13, the target zone 140 is formed by removing an area or portion of the reflective layer 142 at a desired location or, alternatively, by masking the desired area prior to applying the reflective layer 142. As further illustrated in FIG. 13, the plastic adhesive member 118 is applied over the active layer 144. FIG. 13 also shows the cap portion 116 and the reflective surface 146 associated therewith. Thus, when the cap portion 116 is applied to the plastic adhesive member 118 including the desired cutout shapes, the flow channel 130 is thereby formed.

FIG. 14 is a cross sectional view taken across the tracks of the transmissive disc embodiment of the bio-disc 110 according to the present invention as described in FIG. 12, for example. This view is taken longitudinally along a radius and flow channel of the disc. FIG. 14 illustrates the substrate 120 and the thin semi-reflective layer 143. This thin semi-reflective layer 143 allows the incident or interrogation beam 152, from the light source 150, to penetrate and pass through the disc to be detected by the top detector 158, while some of the light is reflected back in the form of the return beam 154. The thickness of the thin semi-reflective layer 143 is determined by the minimum amount of reflected light required by the disc reader to maintain its tracking ability. The substrate 120 in this embodiment, like that discussed in FIG. 13, includes the series of grooves 170. The grooves 170 in this embodiment are also preferably in the form of a spiral extending from near the center of the disc toward the outer edge. The grooves 170 are implemented so that the interrogation beam 152 may track along the spiral. FIG. 14 also shows the active layer 144 applied over the thin semi-reflective layer 143. As further illustrated in FIG. 14, the plastic adhesive member or channel layer 118 is applied over the active layer 144. FIG. 14 also shows the cap portion 116 without a reflective surface 146. Thus, when the cap is applied to the plastic adhesive member 118 including the desired cutout shapes, the flow channel 130 is thereby formed and a part of the incident beam 152 is allowed to pass therethrough substantially unreflected.

FIG. 15 is a view similar to FIG. 11 showing the entire thickness of the reflective disc and the initial refractive property thereof. FIG. 16 is a view similar to FIG. 12 showing the entire thickness of the transmissive disc and the initial refractive property thereof. Grooves 170 are not seen in FIGS. 15 and 16 since the sections are cut along the grooves 170. FIGS. 15 and 16 show the presence of the narrow flow channel 130 that is situated perpendicular to the grooves 170 in these embodiments. FIGS. 13, 14, 15, and 16 show the entire thickness of the respective reflective and transmissive discs. In these figures, the incident beam 152 is illustrated initially interacting with the substrate 120 which has refractive properties that change the path of the incident beam as illustrated to provide focusing of the beam 152 on the reflective layer 142 or the thin semi-reflective layer 143.

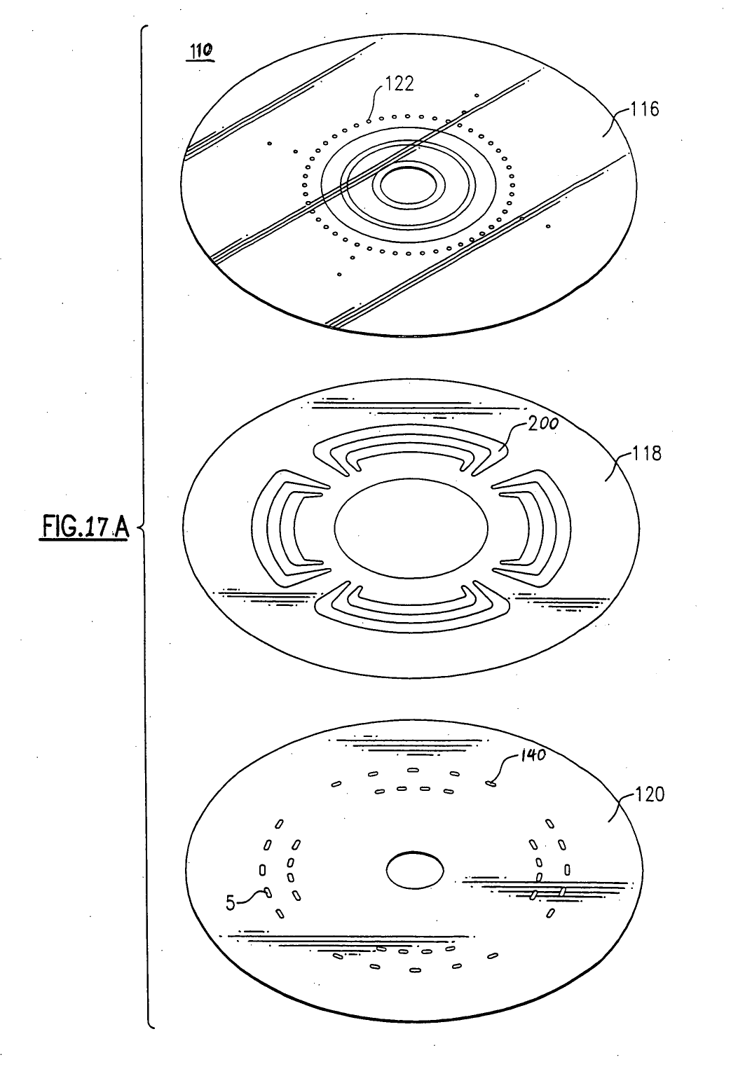

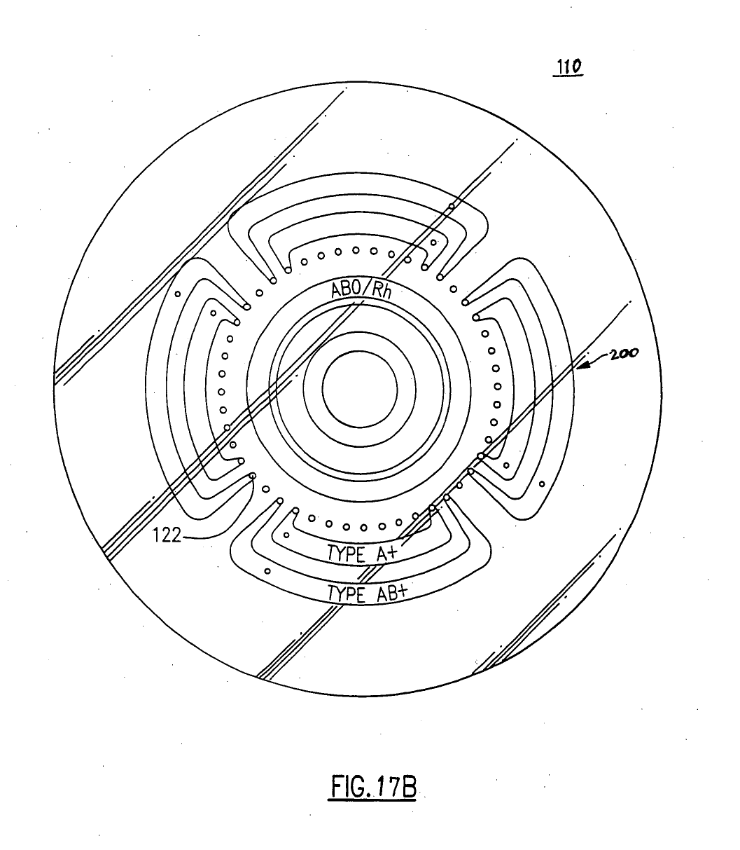

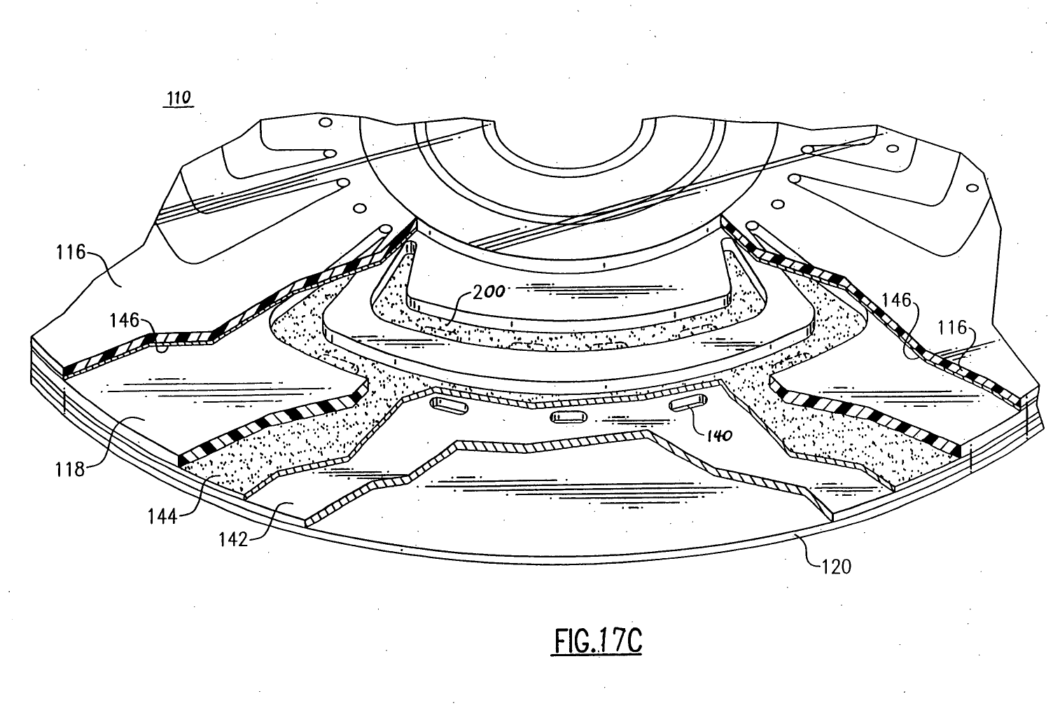

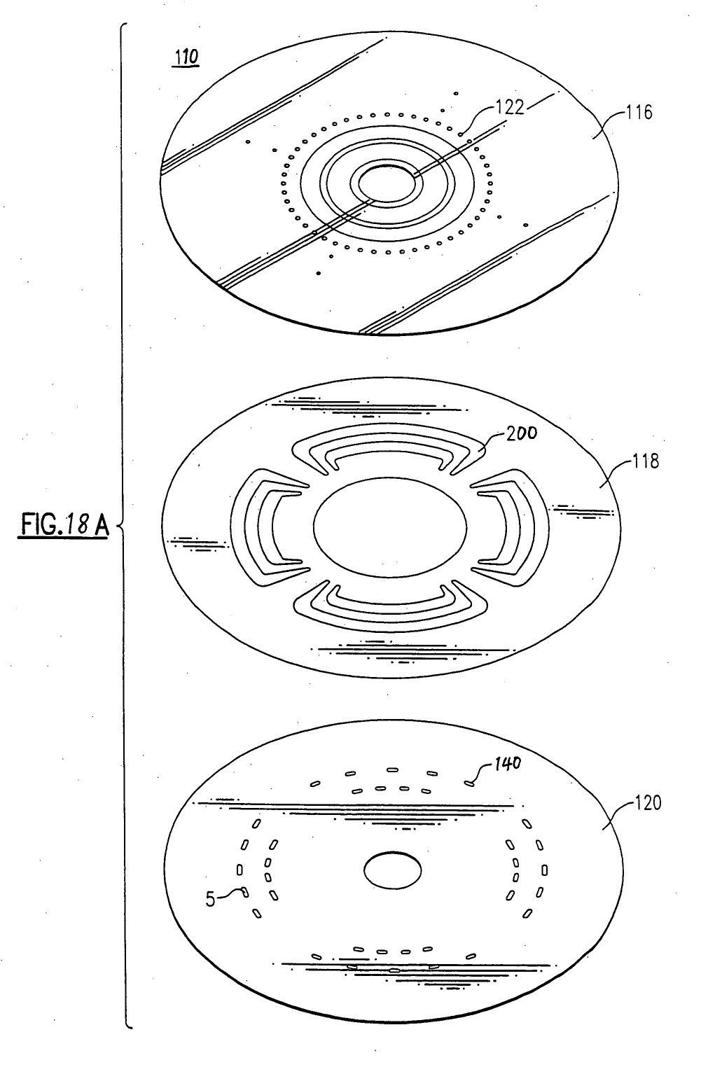

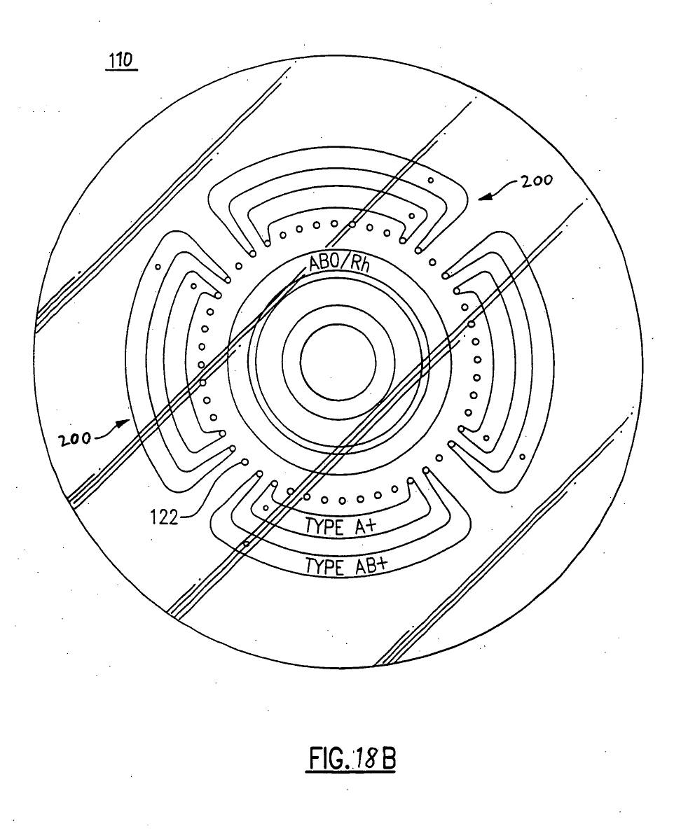

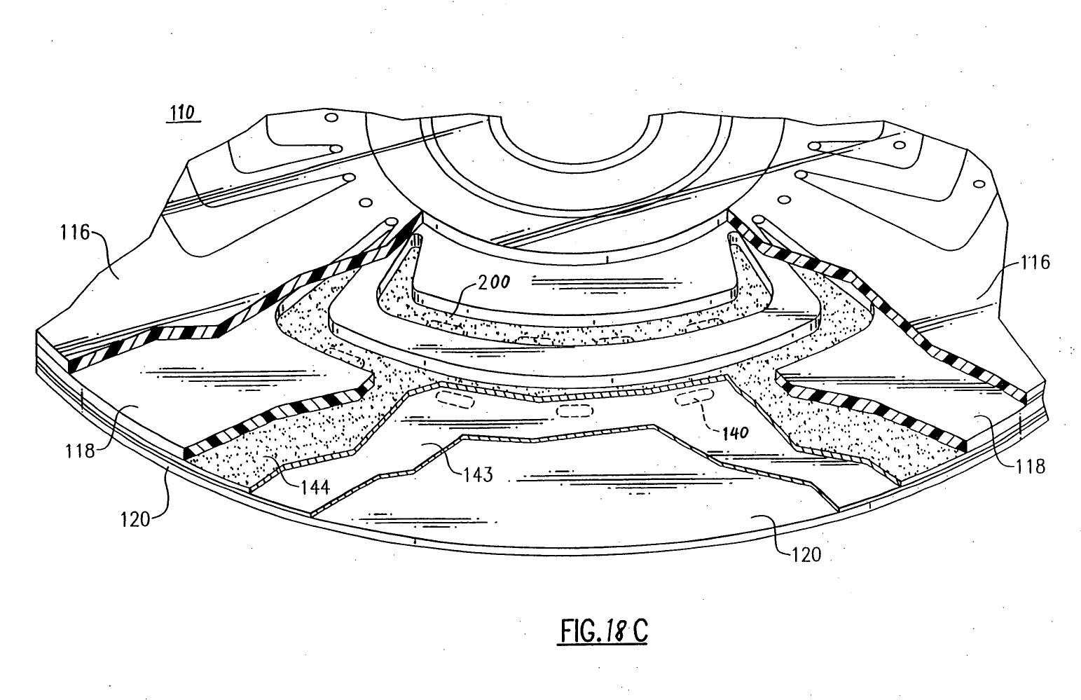

Alternative embodiments of the bio-disc according to the present invention will now be described with reference to FIGS. 17A, 17B, 17C, 18A, 18B, and 18C. Various features of the discs of these latter embodiments have been already illustrated with reference to FIGS. 1 to 16, and therefore such common features will not be described again in the following. Accordingly, and for the sake of simplicity, as a general rule in FIGS. 17 and 18, only the features differentiating the bio-disc 110 from those of FIGS. 1 to 21 are represented.

Furthermore, the following description of the bio-disc of the invention can be readily applied to a transmissive-type as well as to a reflective-type optical bio-disc described above in conjunction with FIGS. 2 to 9.

FIG. 17A is an exploded perspective view of a reflective bio-disc incorporating equi-radial channels 200 of the present invention. This general construction corresponds to the radial-channel disc shown in FIG. 2. The e-rad or eRad implementation of the bio-disc 110 shown in FIG. 17A similarly includes the cap 116, the channel layer 118, and the substrate 120. The channel layer 118 includes the equi-radial fluid channels 200, while the substrate 120 includes the corresponding arrays of target zones 140.

FIG. 17B is a top plan view of the disc shown in FIG. 17A. FIG. 17B further shows a top plan view of an embodiment of eRad disc with a transparent cap portion, which disc has two tiers of circumferential fluid channels with ABO chemistry and two blood types (A+ and AB+). As shown in FIG. 17B, it is also possible to provide a priori, at the manufacturing stage of the disc of the invention, a plurality of entry ports, eventually at different radial coordinate, so that a range of equi-radial, spiralling, or radial reaction sites and/or channels are possible on one disc. These channels can be used for different test suites, or for multiple samples of single test suites.

FIG. 17C is a perspective view of the disc illustrated in FIG. 17A with cut-away sections showing the different layers of the e-radial reflective disc. This view is similar to the reflective disc shown in FIG. 4. The e-rad implementation of the reflective bio-disc shown in FIG. 17C similarly includes the reflective layer 142, active layer 144 as applied over the reflective layer 142, and the reflective layer 146 on the cap portion 116.

FIG. 18A is an exploded perspective view of a transmissive bio-disc utilizing the e-radial channels of the present invention. This general construction corresponds to the radial-channel disc shown in FIG. 5. The transmissive e-rad implementation of the bio-disc 110 shown in FIG. 18A similarly includes the cap 116, the channel layer 118, and the substrate 120. The channel layer 118 includes the equi-radial fluid channels 200, while the substrate 120 includes the corresponding arrays of target zones 140.

FIG. 18B is a top plan view of the transmissive e-rad disc shown in FIG. 18A. FIG. 18B further shows two tiers of circumferential fluid channels with ABO chemistry and two blood types (A+ and AB+). As previously discussed, the assays are performed in the target, capture, or analysis zones 140.

FIG. 18C is a perspective view of the disc illustrated in FIG. 18A with cut-away sections showing the different layers of this embodiment of the e-rad transmissive bio-disc. This view is similar to the transmissive disc shown in FIG. 9. The e-rad implementation of the transmissive bio-disc shown in FIG. 18C similarly includes the thin semi-reflective layer 143 and the active layer 144 as applied over the thin semi-reflective layer 143.

Detection of Hemoglobin and Glycohemoglobin Using the Optical Bio-Disc

Glycohemoglobin analysis is used in long-term carbohydrate control of diabetics. Glycohemoglobin is formed when glucose binds to hemoglobin (Hb) at the N-terminal valine on the beta-chain resulting in the formation of HbAlc. Antibody-based assays have been used to detect the non-enzymatic glycation of Hb directly. However, producing HbAlc specific antibodies in animals is very difficult since the sugar moiety of the glycohemoglobin molecule is not exposed and will rarely result in a specific immuneresponse. A combination of isocratic ion exchange chromatography with a class-specific immunoassay for hemoglobin can rapidly analyze glycated hemoglobin without the need of a specific probe for HbAlc. Different methods for glycohemoglobin analysis implemented on the optical bio-discs are described below.

Cation Exchange Linked Immunoassay (CELIA) on the Optical Bio-Disc Ion Exchange Resins

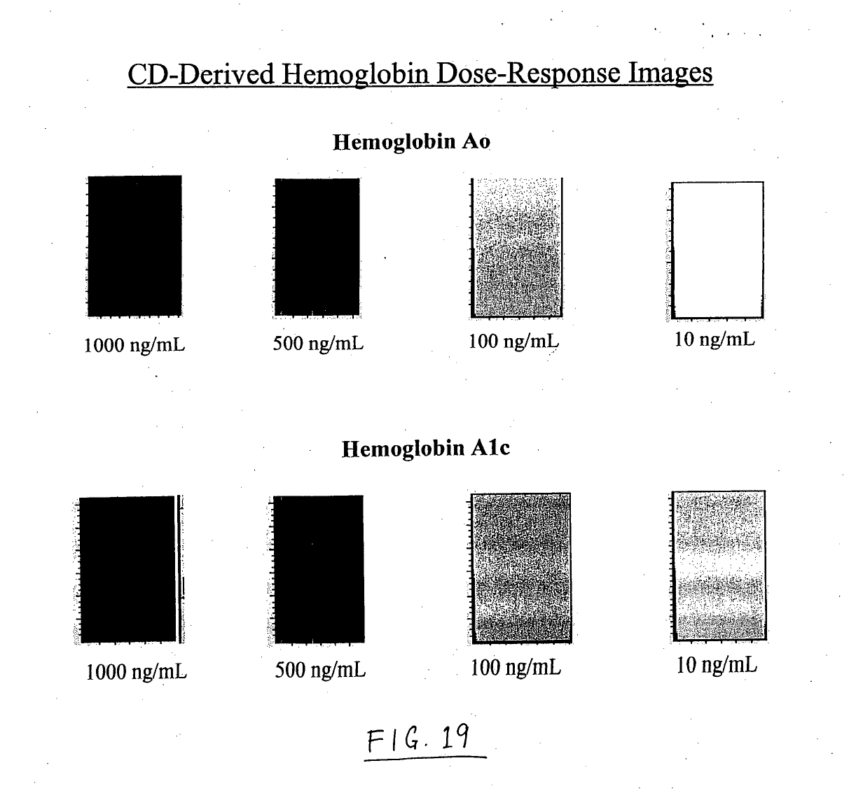

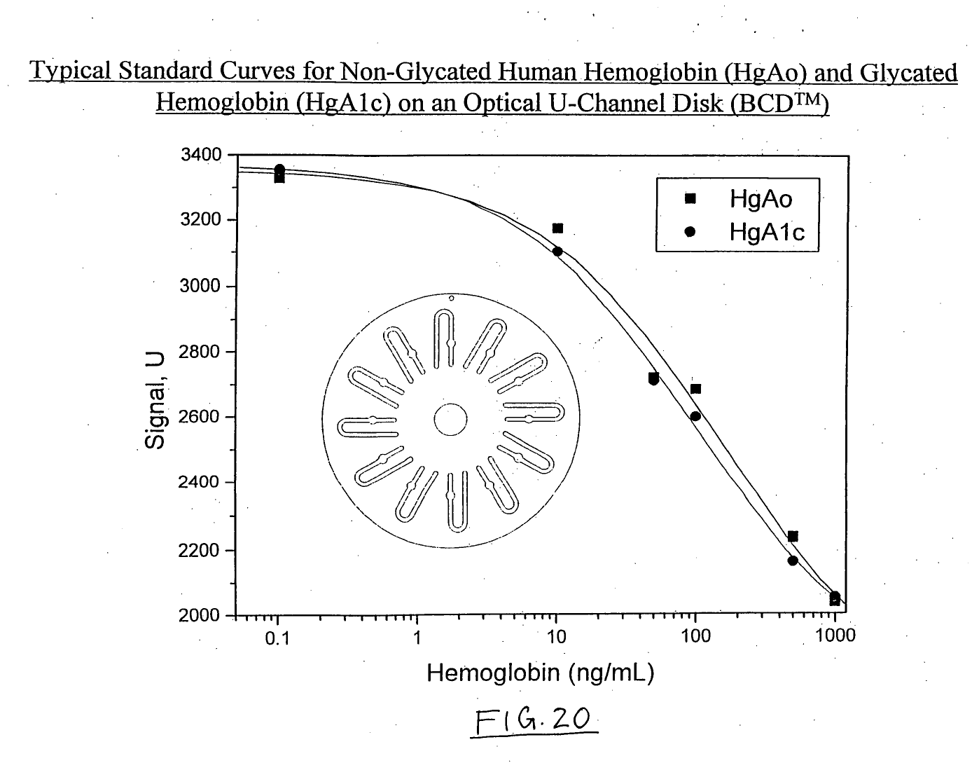

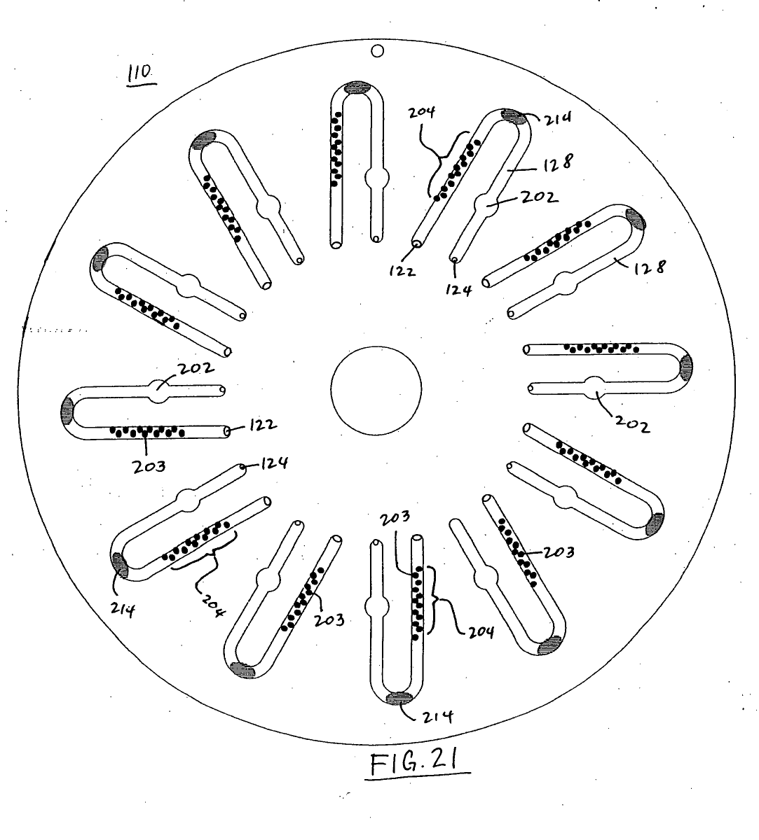

A sandwich immunoassay for hemoglobin was developed by immobilizing haptoglobin (a general capture agent for hemoglobin species) directly on the gold surface of an optical bio-disc substrate. Horseradish peroxidase (HRP)-labeled goat anti-human hemoglobin antibody was used as the enzyme conjugated signal antibody. ABTS (2,2′-azino-di-(3-ethyl-benzthiazoline sulfonic acid) was used as the enzyme substrate. Optical bio-disc derived images were taken and four-parameter-fitted standard curves were generated as shown in FIGS. 19 and 20. The results indicate that the optical bio-disc assay is sensitive for hemoglobin and is capable of detecting both glycated and non-glycated hemoglobin species to the same degree.

Weak cation exchange resins (e.g., carboxymethyl Sephadex beads) may be used to separate non-glycated hemoglobin from glycated hemoglobin species in a test sample. FIG. 21 illustrates an embodiment of the optical bio-disc of the present invention wherein weak cation exchange beads 203 are integrated into the fluidic circuit 128 to form a micro-chromatographic matrix 204 in the optical bio-disc 110 to isolate desired analytes including glycated hemoglobin, for example. In this method, a hemoglobin sample (e.g. blood lysate), containing both glycated and non-glycated forms of hemoglobin, is loaded into the inlet port 122. The disc 110 is then spun thereby moving the sample through the cation exchange micro-chromatographic matrix 204. The non-glycated hemoglobin binds to the beads 203 and only the glycated hemoglobin leaves the matrix 204 and moves through a filter 214 and into an analysis or assay zone 202 where the analyte is quantified as described above. Alternatively, the non-glycated hemoglobin may be isolated using anionic beads. In this alternative embodiment, glycated hemoglobin bind to the anionic beads while the non-glycated hemoglobin passes through the micro-chromatographic matrix 204 and is quantified. The total hemoglobin also needs to be quantified along with either the glycated or non-glycated hemoglobin to determine the percentage of glycated hemoglobin. The total hemoglobin may be quantified directly using the sample loaded directly into the analysis zone 202 or neutral beads may also be used in the micro-chromatographic matrix 204 wherein both forms of hemoglobin can freely pass through thereby allowing quantitation of the total hemoglobin.

Fluorescent labels may be used instead of HRP-labeled anti human hemoglobin signal antibodies and the assay quantified using a fluorescent optical bio-disc drive. Furthermore the capture and signal agents may be haptoglobin instead of antibodies. In this case, the assay will consist of a haptoglobin capture agent immobilized on a capture or target zone within an analysis chamber and a HRP- or fluorescent labeled haptoglobin signal agent. Other detectable labels known in the art can also be applied. The pseudo-peroxidase activity of hemoglobin can also be used to produce a detectable signal with the appropriate peroxidase substrate and requires only the (unlabeled) haptoglobin capture agent (or other capture proteins for hemoglobin) to capture the analyte, as described above.

The ion exchange matrix may be packed into the fluidic channels and separated from the analysis chamber 202 by using a different channel and/or chamber thickness for the analysis chamber 202. For example, 40-120 micron cation exchange beads may be used to form the ion exchange matrix. Thus a channel or chamber on the disc with a thickness of >120 microns (“ion exchange zone”) connected to a second channel or chamber with a thickness of <40 microns (analysis chamber) can be used. The narrower thickness of the analysis chamber prevents the beads from entering the analysis chamber. Furthermore a microfluidic channel design with a capillary valve system can also be used in conjunction with the ion exchange linked immunoassay embodiments of the present invention.

Ion Exchange Membranes

1) Lateral Flow Membranes

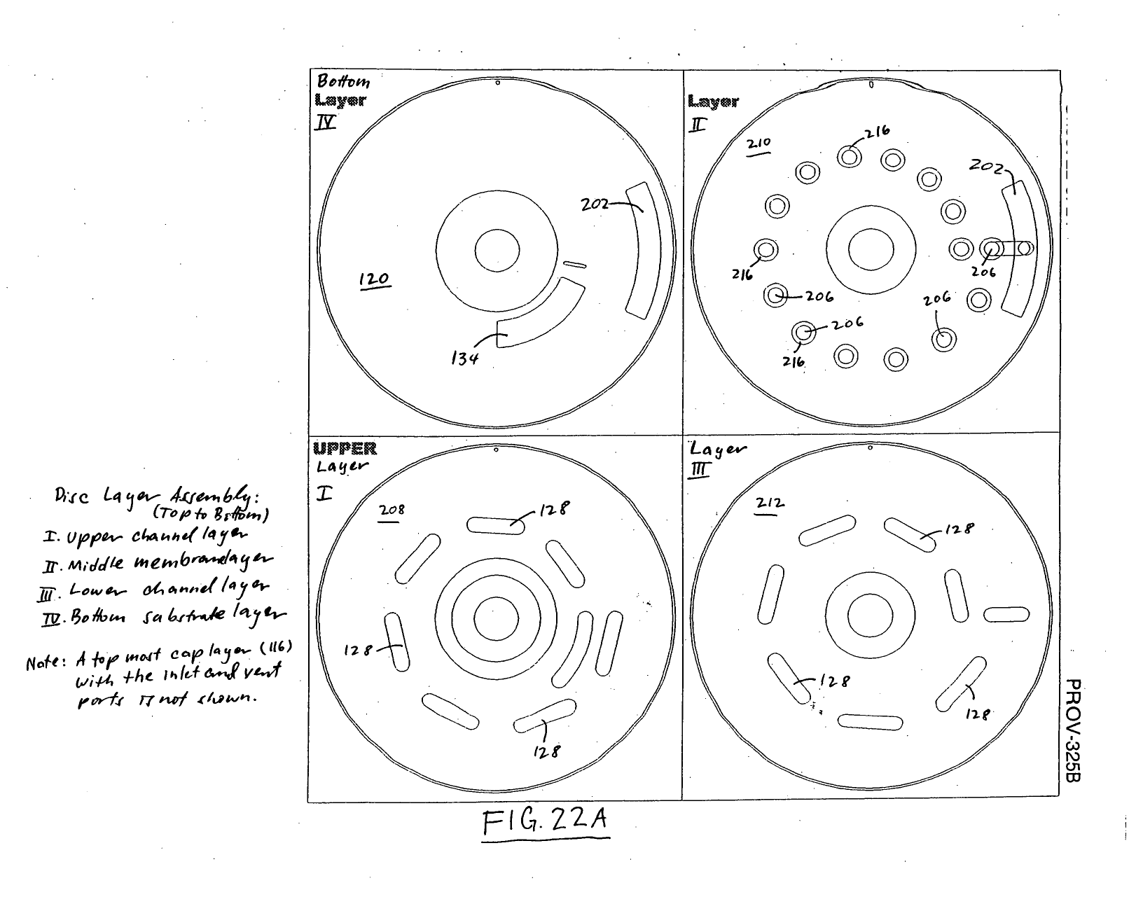

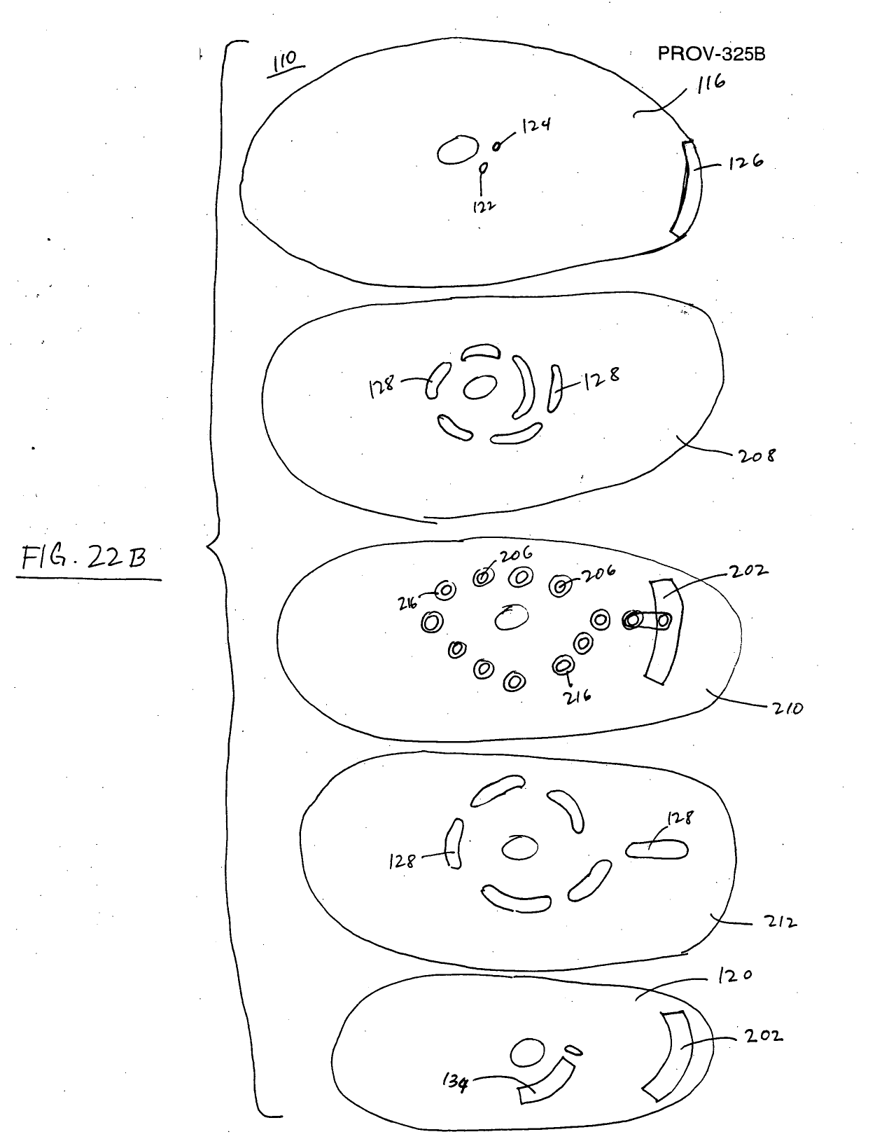

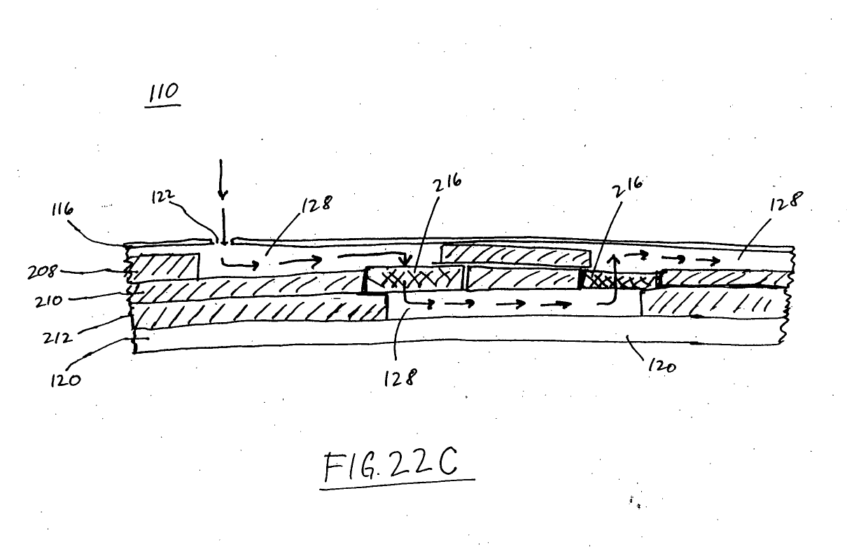

FIGS. 22A, 22B, and 22C show an optical bio-disc 110 for use in the membrane chromatographic assay of the present invention wherein chemically modified membranes 216 having binders directed to either glycated or non-glycated hemoglobin, for example, may be used as the matrix material of the present invention. In this case the lateral flow membrane 216 may be formed, for example, from carboxymethyl (a weak cation) membranes, for binding non-glycated hemoglobin.

In a sandwich assay format method of the present invention, the capture agent, which can be an antibody or haptoglobin or another capture protein for hemoglobin, may be labeled with reporter particles (latex beads, gold beads, carbon beads, or others). After sample application and disc spinning steps, non-glycated hemoglobin binds to the cation exchange matrix and glycated hemoglobin will move to the specific analysis chamber and to the target or capture zone. The target zone is then analyzed for the presence and amount of reporter particles using the optical bio-disc reader. For the measurement of non-glycated hemoglobin the ion exchange matrix may be formed from a weak anion exchange membrane.

2) Flow Through Membrane (Membrane Adsorbers)

Ion Exchange Membrane Adsorbers used in ready-to-use filters (Sartorius, Goettengen, Germany) may also be used to form the matrix. Furthermore, centrifuge based Ion Exchange Membrane Spin Columns such as for example Vivapure (Viva Science, Hannover, Germany) can also be embedded into an optical bio-disc and used for the separation of different isoforms of proteins (including various hemoglobin species) with subsequent, immunoassay-based optical bio-disc detection.

With reference to FIG. 22A, there is shown different layers of the bio-disc 110 for use in the lateral flow and flow through membrane based assays of the present invention. In this embodiment, several layers may be assembled to form the spiral fluidic circuit 128 as shown. These layers may include a top cover disc or cap portion 116 (illustrated in FIG. 22B), an upper channel layer 208, a lower channel layer 212, a middle membrane or chromatography layer 210 situated between upper layer 208 and lower layer 212, and a bottom substrate layer 120. Substrate layer 120 may be the transmissive or reflective type substrate 120 as discussed above. The top cap portion 116 includes one or more inlet ports 122 and one or more vent ports 124 as shown in FIGS. 2, 5, 17A, and 22B. The chromatography layer 210 includes pass through ports 206 formed therein. The chemically modified membranes 216 are may be placed over the pass through ports 206. The upper 208 and lower 212 channel layers have fluidic circuits 128 formed therein such that when the disc 110 is assembled with the chromatography layer 210 placed between the upper 208 and lower 212 channel layers, and the bottom substrate layer 120 and top cap portion 116 are accordingly bonded to the disc; a spiral fluidic chromatographic circuit is formed.

Referring now to FIG. 22B, there is depicted an exploded view of the bio-disc 110 described above in conjunction with FIG. 22A showing the various layers of the bio-disc including the top cap portion 116, the upper channel layer 208, the chromatography layer 210, the lower channel layer 212, and the bottom substrate layer 120.

Turning next to FIG. 22C, there is illustrated a partial cross section of a fully assembled bio-disc as described in FIG. 22A showing the direction of fluid flow (arrows) through the fluidic circuit 128. Sample is introduced into the disc 110 through the inlet port 122 of the cap portion 116. The upper channel layer 208, chromatography layer 210, and lower channel layer 212 are positioned such that fluid is directed through a series of chemically modified membranes 216 as the fluid or sample moves through the fluidic circuit 128 as illustrated. The chemically modified membrane 216 may include for example the Ion Exchange and Lateral Flow Membranes described above.

Bioseparation with a porous membrane is of critical importance in molecular biology assays. The present application demonstrates fluidic channel arrangements for integration of porous materials, such as a porous membrane or a chromatographic membrane, into the optical bio-disc 110.

The bio-disc 110 is preferably made from several layers of polycarbonate discs and patterned adhesives to form a fluidic circuit. By integrating the porous membrane, the applied analyte will flow through the porous material when the analyte is driven by centrifugal and/or other types of forces.

With continuing reference to FIG. 22C, there is shown a pattern for each layer of a disc for use in biochemical assays. The optical bio-disc 110 of the present invention may include the following layers:

1. Substrate Layer 120 is a lens disc with signal tracks. The substrate layer may be a CD, CD-R, DVD, or DVD-R type disc, for example. The substrate 120 may include a reflective layer 142 which can be transmissive or partially reflective as described above in conjunction with FIGS. 2-9. Thus, it can be used to track disc spinning and provide enough optical signal for detection.

2. Lower channel layer 212 may be formed from an adhesive with fluidic channels 128 formed therein.

3. Chromatographic layer 210 is a disc layer having pass through ports 206 designed such that a chromatographic membrane material 216 may be integrated into the optical bio-disc 110. Chromatographic membranes 216 are preferably placed in or on the pass through ports 206. The membrane and chromatographic layer thickness are preferably identical. If the thickness of the membrane and chromatographic layer is different, then thickness of each can be adjusted by applying multiple layers.

4. Upper channel layer 208 may be formed from an adhesive with fluidic channels formed therein. The patterned fluidic channels overlap with the fluidic channels from the lower channel layer 212 at the pass through ports 206 of the chromatographic layer only, as shown. Thus, the analyte will pass through these fluidic paths by vertically flowing through the membranes only, as shown in FIG. 22C

5. The topmost cap portion 116 is a cover disc. The fluidic channels 128 are made to accommodate the test sample, especially when a large analyte volume is required for the assay.

6. The optical bio-disc of the present invention may optionally include a sealing layer (not shown) over the cap portion 116. It covers the vent port 124 and inlet port 122 and prevents contamination of the fluidic circuits 128 and also prevents evaporation of the test sample when loaded into the bio-disc.

Generally, the separation concept is based on having the chromatographic membrane material 216 arranged within the two layers of fluidic path as shown in FIG. 22C. Furthermore, bioseparation can be achieved by properly arranging the fluidic path to allow the analyte to flow through a series of chromatographic membranes 216.

FIG. 22C shows one segment of the integration arrangement and this design module can be scaled-up or scaled-down, by considering such factors as: membrane size, numbers of membrane required, and required disc space.

By extending this module in series, the analyte can flow through more than two layers of membrane (as shown in FIG. 22). The present invention may be used for hemoglobin separation using a cation exchange membrane. The present invention may also be used in various bioseparation applications which are different from separation by porous sizing only.

More particular discussion of membranes as implemented on optical bio-discs are provided in the following commonly assigned and co-pending provisional applications: U.S. Provisional App. Ser. No. 60/353,740 entitled “Methods and Apparatus for Separation of Lipoproteins Using Membranes on Optical Bio-Discs” filed Jan. 30, 2002; U.S. Provisional App. Ser. No. 60/353,300 entitled “Methods for Differential Cell Counts Including Leukocytes and Use of Optical Bio-Disc for Performing Same” filed Jan. 31, 2002; U.S. Provisional App. Ser. No. 60/353,948 entitled “Methods for Quantitation and Multiplexing of Receptor Ligand Assay by Use of Ultrathin Biomembranes Including Modified Optical Disc and Drive” filed Jan. 31, 2002; U.S. Provisional App. Ser. No. 60/354,014 entitled “Membrane Assays Implemented On Optical Analysis Disc” filed Jan. 31, 2002; U.S. Provisional App. Ser. No. 60/353,913 entitled “Optical Bio-Disc Membrane Quantification Apparatus and Methods Using Control Lines as Internal Standard” filed Jan. 31, 2002; U.S. Provisional App. Ser. No. 60/353,818 entitled “Methods And Apparatus For Separation Of Blood Using Membranes On Optical Bio-Discs” filed Jan. 31, 2002; U.S. Provisional App. Ser. No. 60/354,319 entitled “Use of Avidin-Biotin Systems for Increase Detection Sensitivity in Membrane Based Assays and Related Optical Analysis Disc” filed Feb. 4, 2002; and U.S. Provisional App. Ser. No. 60/354,379 entitled “Application Methods For Bio-Membrane Assays In Bio-Disc System and Optical Analysis Disc Made According Thereto” filed Feb. 4, 2002. All of these applications are herein incorporated by reference in their entireties. They thus provide background and related disclosure as support hereof as if fully repeated herein.

Additional embodiments, aspects, details, and attributes of the present invention are disclosed in Appendix A and B, appended hereto. These appendices are therefore a part hereof wherein more specifically Appendix A includes pages A1-A38, and Appendix B includes pages B1-B23.

3) Other Implementations of the Current Invention

This invention or different aspects thereof may be readily implemented in, adapted to, or employed in combination with the discs, assays, and systems disclosed in the following commonly assigned and co-pending patent applications: U.S. patent application Ser. No. 09/378,878 entitled “Methods and Apparatus for Analyzing Operational and Non-operational Data Acquired from Optical Discs” filed Aug. 23, 1999; U.S. Provisional Patent Application Ser. No. 60/150,288 entitled “Methods and Apparatus for Optical Disc Data Acquisition Using Physical Synchronization Markers” filed Aug. 23, 1999; U.S. patent application Ser. No. 09/421,870 entitled “Trackable Optical Discs with Concurrently Readable Analyte Material” filed Oct. 26, 1999; U.S. patent application Ser. No. 09/643,106 entitled “Methods and Apparatus for Optical Disc Data Acquisition Using Physical Synchronization Markers” filed Aug. 21, 2000; U.S. patent application Ser. No. 09/999,274 entitled “Optical Biodiscs with Reflective Layers” filed Nov. 15, 2001; U.S. patent application Ser. No. 09/988,728 entitled “Methods and Apparatus for Detecting and Quantifying Lymphocytes with Optical Biodiscs” filed Nov. 16, 2001; U.S. patent application Ser. No. 09/988,850 entitled “Methods and Apparatus for Blood Typing with Optical Bio-discs” filed Nov. 19, 2001; U.S. patent application Ser. No. 09/989,684 entitled “Apparatus and Methods for Separating Agglutinants and Disperse Particles” filed Nov. 20, 2001; U.S. patent application Ser. No. 09/997,741 entitled “Dual Bead Assays Including Optical Biodiscs and Methods Relating Thereto” filed Nov. 27, 2001; U.S. patent application Ser. No. 09/997,895 entitled “Apparatus and Methods for Separating Components of Particulate Suspension” filed Nov. 30, 2001; U.S. patent application Ser. No. 10/005,313 entitled “Optical Discs for Measuring Analytes” filed Dec. 7, 2001; U.S. patent application Ser. No. 10/006,371 entitled “Methods for Detecting Analytes Using Optical Discs and Optical Disc Readers” filed Dec. 10, 2001; U.S. patent application Ser. No. 10/006,620 entitled “Multiple Data Layer Optical Discs for Detecting Analytes” filed Dec. 10, 2001; U.S. patent application Ser. No. 10/006,619 entitled “Optical Disc Assemblies for Performing Assays” filed Dec. 10, 2001; U.S. patent application Ser. No. 10/020,140 entitled “Detection System For Disk-Based Laboratory and Improved Optical Bio-Disc Including Same” filed Dec. 14, 2001; U.S. patent application Ser. No. 10/035,836 entitled “Surface Assembly for Immobilizing DNA Capture Probes and Bead-Based Assay Including Optical Bio-Discs and Methods Relating Thereto” filed Dec. 21, 2001; U.S. patent application Ser. No. 10/038,297 entitled “Dual Bead Assays Including Covalent Linkages for Improved Specificity and Related Optical Analysis Discs” filed Jan. 4, 2002; U.S. patent application Ser. No. 10/043,688 entitled “Optical Disc Analysis System Including Related Methods for Biological and Medical Imaging” filed Jan. 10, 2002; U.S. Provisional Application Ser. No. 60/348,767 entitled “Optical Disc Analysis System Including Related Signal Processing Methods and Software” filed Jan. 14, 2002 U.S. patent application Ser. No. 10/086,941 entitled “Methods for DNA Conjugation Onto Solid Phase Including Related Optical Biodiscs and Disc Drive Systems” filed Feb. 26, 2002; U.S. patent application Ser. No. 10/087,549 entitled “Methods for Decreasing Non-Specific Binding of Beads in Dual Bead Assays Including Related Optical Biodiscs and Disc Drive Systems” filed Feb. 28, 2002; U.S. patent application Ser. No. 10/099,256 entitled “Dual Bead Assays Using Cleavable Spacers and/or Ligation to Improve Specificity and Sensitivity Including Related Methods and Apparatus” filed Mar. 14, 2002; U.S. patent application Ser. No. 10/099,266 entitled “Use of Restriction Enzymes and Other Chemical Methods to Decrease Non-Specific Binding in Dual Bead Assays and Related Bio-Discs, Methods, and System Apparatus for Detecting Medical Targets” also filed Mar. 14, 2002; U.S. patent application Ser. No. 10/121,281 entitled “Multi-Parameter Assays Including Analysis Discs and Methods Relating Thereto” filed Apr. 11, 2002; U.S. patent application Ser. No. 10/150,575 entitled “Variable Sampling Control for Rendering Pixelization of Analysis Results in a Bio-Disc Assembly and Apparatus Relating Thereto” filed May 16, 2002; U.S. patent application Ser. No. 10/150,702 entitled “Surface Assembly For Immobilizing DNA Capture Probes in Genetic Assays Using Enzymatic Reactions to Generate Signals in Optical Bio-Discs and Methods Relating Thereto” filed May 16, 2002; U.S. patent application Ser. No. 10/194,418 entitled “Optical Disc System and Related Detecting and Decoding Methods for Analysis of Microscopic Structures” filed Jul. 12, 2002; U.S. patent application Ser. No. 10/194,396 entitled “Multi-Purpose Optical Analysis Disc for Conducting Assays and Various Reporting Agents for Use Therewith” also filed Jul. 12, 2002; U.S. patent application Ser. No. 10/199,973 entitled “Transmissive Optical Disc Assemblies for Performing Physical Measurements and Methods Relating Thereto” filed Jul. 19, 2002; U.S. patent application Ser. No. 10/201,591 entitled “Optical Analysis Disc and Related Drive Assembly for Performing Interactive Centrifugation” filed Jul. 22, 2002; U.S. patent application Ser. No. 10/205,011 entitled “Method and Apparatus for Bonded Fluidic Circuit for Optical Bio-Disc” filed Jul. 24, 2002; U.S. patent application Ser. No. 10/205,005 entitled “Magnetic Assisted Detection of Magnetic Beads Using Optical Disc Drives” also filed Jul. 24, 2002; U.S. patent application Ser. No. 10/230,959 entitled “Methods for Qualitative and Quantitative Analysis of Cells and Related Optical Bio-Disc Systems” filed Aug. 29, 2002; U.S. patent application Ser. No. 10/233,322 entitled “Capture Layer Assemblies for Cellular Assays Including Related Optical Analysis Discs and Methods” filed Aug. 30, 2002; U.S. patent application Ser. No. 10/236,857 entitled “Nuclear Morphology Based Identification and Quantification of White Blood Cell Types Using Optical Bio-Disc Systems” filed Sep. 6, 2002; U.S. patent application Ser. No. 10/241,512 entitled “Methods for Differential Cell Counts Including Related Apparatus and Software for Performing Same” filed Sep. 11, 2002; U.S. patent application Ser. No. 10/279,677 entitled “Segmented Area Detector for Biodrive and Methods Relating Thereto” filed Oct. 24, 2002; U.S. patent application Ser. No. 10/293,214 entitled “Optical Bio-Discs and Fluidic Circuits for Analysis of Cells and Methods Relating Thereto” filed on Nov. 13, 2002; U.S. patent application Ser. No. 10/298,263 entitled “Methods and Apparatus for Blood Typing with Optical Bio-Discs” filed on Nov. 15, 2002; U.S. patent application Ser. No. 10/307,263 entitled “Magneto-Optical Bio-Discs and Systems Including Related Methods” filed Nov. 27, 2002; U.S. patent application Ser. No. 10/341,326 entitled “Method and Apparatus for Visualizing Data” filed Jan. 13, 2003; U.S. patent application Ser. No. 10/345,122 entitled “Methods and Apparatus for Extracting Data From an Optical Analysis Disc” filed on Jan. 14, 2003; U.S. patent application Ser. No. 10/347,155 entitled “Optical Discs Including Equi-Radial and/or Spiral Analysis Zones and Related Disc Drive Systems and Methods” filed on Jan. 15, 2003; U.S. patent application Ser. No. 10/347,119 entitled “Bio-Safe Dispenser and Optical Analysis Disc Assembly” filed Jan. 17, 2003; U.S. patent application Ser. No. 10/348,049 entitled “Multi-Purpose Optical Analysis Disc for Conducting Assays and Related Methods for Attaching Capture Agents” filed on Jan. 21, 2003; U.S. patent application Ser. No. 10/348,196 entitled “Processes for Manufacturing Optical Analysis Discs with Molded Microfluidic Structures and Discs Made According Thereto” filed on Jan. 21, 2003; U.S. patent application Ser. No. 10/351,604 entitled “Methods for Triggering Through Disc Grooves and Related Optical Analysis Discs and System” filed on Jan. 23, 2003; U.S. patent application Ser. No. 10/351,280 entitled “Bio-Safety Features for Optical Analysis Discs and Disc System Including Same” filed on Jan. 23, 2003; U.S. patent application Ser. No. 10/351,244 entitled “Manufacturing Processes for Making Optical Analysis Discs Including Successive Patterning Operations and Optical Discs Thereby Manufactured” filed on Jan. 24, 2003; U.S. patent application Ser. No. 10/353,777 entitled “Processes for Manufacturing Optical Analysis Discs with Molded Microfluidic Structures and Discs Made According Thereto” filed on Jan. 27, 2003; U.S. patent application Ser. No. 10/353,839 entitled “Method and Apparatus for Logical Triggering” filed on Jan. 28, 2003; and U.S. patent application Ser. No. 10/356,666 entitled “Methods For Synthesis of Bio-Active Nanoparticles and Nanocapsules For Use in Optical Bio-Disc Assays and Disc Assembly Including Same” filed Jan. 30, 2003. All of these applications are herein incorporated by reference in their entireties. They thus provide background and related disclosure as support hereof as if fully repeated herein.

### CONCLUDING SUMMARY

All patents, provisional applications, patent applications, technical specifications, and other publications mentioned in this specification are incorporated herein in their entireties by reference.

While this invention has been described in detail with reference to certain preferred embodiments, it should be appreciated that the present invention is not limited to those precise embodiments. Rather, in view of the present optical bio-system disclosure that describes the current best mode for practicing the invention, many modifications and variations would present themselves to those of skill in the art without departing from the scope and spirit of this invention. The scope of the invention is, therefore, indicated by the following claims rather than by the foregoing description. All changes, modifications, and variations coming within the meaning and range of equivalency of the claims are to be considered within their scope.

Furthermore, those skilled in the art will recognize, or be able to ascertain, using no more than routine experimentation, many equivalents to the specific embodiments of the invention described herein. Such equivalents are also intended to be encompassed by the following claims.

1. A chromatographic optical bio-disc, comprising:
a cap portion having inlet and vent ports formed therein;
a first channel layer having a first set of flow channels formed therein;
a chromatographic layer having pass through ports formed therein;
a second channel layer having a second set of flow channels formed therein; and
a substantially circular substrate having a center and an outer edge.

2. The optical bio-disc according to claim 1 further comprising a target zone disposed between the center and the outer edge of said substrate in fluid communication with said first set of flow channels, said second set of flow channels, and said pass through ports.

3. The optical bio-disc according to claim 2 further comprising capture agents located within said target zone.

4. The optical bio-disc according to claim 3 further comprising chromatography membranes.

5. The optical bio-disc according to claim 4 wherein said chromatography membranes are placed on said pass through ports such that when a sample is introduced through the inlet port, the sample moves into the first set of flow channels, through the chromatography membranes and the pass through ports, into the second set of flow channels and into the capture zone.

6. The optical bio-disc according to claim 5 wherein said chromatography membranes are ion exchange membranes.

7. The optical bio-disc according to claim 5 wherein said chromatography membranes are membrane adsorbers.

8. The optical bio-disc according to claim 5 wherein said chromatography membranes have binders associated therewith.

9. The optical bio-disc according to claim 8 wherein said binders are directed to glycated hemoglobin.

10. The optical bio-disc according to claim 8 wherein said binders are directed to non-glycated hemoglobin.

11. The optical bio-disc according to claim 1 wherein the substrate includes encoded information associated therewith, the encoded information being readable by a disc drive assembly to control rotation of the bio-disc.

12. The optical bio-disc according to claim 1 further comprising a reflective layer associated with said substrate.

13. The optical bio-disc according to claim 1 further comprising an enzyme, wherein the enzyme, when exposed to an enzyme substrate, produces a signal detectable by an incident beam of electromagnetic radiation.

14. An optical bio-disc, comprising:
a cap portion having inlet and vent ports formed therein;
a channel layer having a fluidic circuit formed therein;
a substantially circular substrate having a center and an outer edge; and
a micro-chromatographic matrix formed in said fluidic circuit.

15. The optical bio-disc of claim 14 further comprising an analysis chamber in fluid communication with said fluidic circuit.

16. The optical bio-disc of claim 15 further comprising a filter placed within said fluidic circuit.

17. The optical bio-disc of claim 16 further comprising a capture agent associated with the substrate in the analysis zone.

18. The optical disc of claim 17 wherein said micro-chromatographic matrix is formed from weak cation exchange beads.

19. The optical disc of claim 17 wherein said micro-chromatographic matrix is formed from anionic beads.

20. The optical bio-disc of claim 18 wherein said capture agent is haptoglobin.

21. The optical bio-disc of claim 20 wherein said analysis chamber is pre-loaded with a signal agent.

22. The optical bio-disc of claim 21 wherein said signal agent is an antibody.

23. The optical bio-disc of claim 22 wherein said antibody is labeled with a tag.

24. The optical bio-disc of claim 23 wherein said tag is detectable by an optical disc reader.

25. The optical bio-disc of claim 23 wherein said tag is selected from the group comprising an enzyme, a fluorescent particle, a fluorescent dye, a luminescent dye, and a luminescent particle.

26. A method of using the optical disc according to claim 24 for testing the amount of hemoglobin Alc in a hemoglobin test sample, said method of using comprising the steps of:
depositing the test sample into the disc through the inlet port;
rotating said disc at a predetermined speed and time to allow said test sample to move through said micro-chromatographic matrix allowing non-glycated hemoglobin present in the sample to bind to said micro-chromatographic matrix;
continuing said rotating step to move said test sample through said filter, and into said analysis chamber;
incubating the test sample to allow any glycated hemoglobin present in the sample to bind with said capture agent and allow said signal agent to bind with said glycated hemoglobin;
washing said analysis chamber to remove unbound signal agents; and
scanning said analysis chamber with a beam of electromagnetic radiation to determine the amount of signal agents bound to the glycated hemoglobin.

27. The method according to claim 26 further comprising the step of calculating the amount of glycated hemoglobin present in the sample based on the amount of bound signal agents.

28. A method of making a chromatographic optical bio-disc, said method comprising the steps of:
providing a substrate having a center and an outer edge;
encoding information on an information layer associated with the substrate, the encoded information being readable by a disc drive assembly to control rotation of the disc;
forming a target zone in association with the substrate, the target zone disposed at a predetermined location relative to the center of the substrate;
depositing a capture agent on the target zone;
forming a flow channel in fluid communication with the target zone; and
forming a micro-chromatographic matrix within the flow channel.

29. A method of making a chromatographic optical bio-disc, said method comprising the steps of:
providing a substrate having a center and an outer edge;
encoding information on an information layer associated with the substrate, the encoded information being readable by a disc drive assembly to control rotation of the disc;
forming a target zone in association with the substrate, the target zone disposed at a predetermined location relative to the center of the substrate;
depositing a capture agent on the target zone;
providing a cap portion having an inlet port and a vent port formed therein;
providing a first channel layer having a first set of flow channels formed therein;
providing a chromatographic layer having pass through ports formed therein;
providing a second channel layer having a second set of flow channels formed therein;
forming a chromatography membrane over said pass through ports; and
assembling the optical bio-disc such that said target zone is in fluid communication with said second set of flow channels, said pass through ports, said first set of flow channels, said inlet port, and said vent port.

30. An optical assay disc implemented to perform any of the methods recited in either claim 26.

31. Use of an optical analysis disc to perform any of the methods recited in either claim 26.

32. An optical disc assembly made to perform any of the methods recited in either claim 26.

33. An optical bio-disc system adapted to operate the optical assay disc recited in claim 30.

34. An optical bio-disc system adapted to read information stored on the optical assay disc recited in claim 30.

35. An optical bio-disc system adapted to write information relating to results of an assay onto the optical assay disc recited in claim 30.

36. An optical bio-disc system adapted to display on a monitor information relating to results of an assay conducted in association with the optical assay disc recited in claim 30.

37. An optical bio-disc system adapted to receive the optical assay disc recited in claim 30 and facilitate the performance of an assay associated with said optical assay disc.

38. An optical bio-disc system adapted to operate the optical analysis disc recited in claim 31.

39. An optical bio-disc system adapted to read information stored on the optical analysis disc recited in claim 31.

40. An optical bio-disc system adapted to write information relating to results of an assay onto the optical analysis disc recited in claim 31.

41. An optical bio-disc system adapted to display on a monitor information relating to results of an assay conducted in association with the optical analysis disc recited in claim 31.

42. An optical bio-disc system adapted to receive the optical analysis disc recited in claim 31 and facilitate the performance of an assay associated with said optical analysis disc.

43. An optical bio-disc system adapted to operate the optical disc assembly recited in claim 32.

44. An optical bio-disc system adapted to read information stored on the optical disc assembly recited in claim 32.

45. An optical bio-disc system adapted to write information relating to results of an assay onto the optical disc assembly recited in claim 32.

46. An optical bio-disc system adapted to display on a monitor information relating to results of an assay conducted in association with the optical disc assembly recited in claim 32.

47. An optical bio-disc system adapted to receive the optical disc assembly recited in claim 32 and facilitate the performance of an assay associated with said optical disc assembly.
